(19) United States
(12) Patent Application Publication
(10) Pub. No.: US 2025/0109379 A1
(43) Pub. Date: Apr. 3, 2025

(54) OPTIMIZATION OF LIVE CELL CONSTRUCTS FOR PRODUCTION OF CULTURED MILK PRODUCT AND METHODS USING THE SAME

(71) Applicant: BIOMILQ, Inc., Durham, NC (US)

(72) Inventors: Akshay Narkhede, Cary, NC (US); Kurt Dornauer, Chapel Hill, NC (US); Leila Strickland, Hillsborough, NC (US); Nour Caroline Dau, Raleigh, NC (US)

(21) Appl. No.: 18/728,774

(22) PCT Filed: Jan. 13, 2023
(86) PCT No.: PCT/US2023/060684

§ 371 Date: Jul. 12, 2024

Related U.S. Application Data
(60) Provisional application No. 63/299,349, filed on Jan. 13, 2022.

(51) Int. Cl. C12N 5/071; A23C 9/20; B82Y 5/00; C12P 1/00

(52) U.S. Cl. CPC C12N 5/0631; A23C 9/206; C12P 1/00; B82Y 5/00; C12N 2501/315; C12N 2513/00; C12N 2533/30; C12N 2533/52; C12N 2533/54

(57) ABSTRACT

Provided herein are cell constructs for producing a cultured milk product from mammary epithelial cells (MECs). In some embodiments, the cell constructs comprise a scaffold, a culture medium in fluidic contact with the scaffold, and mammary cells coupled to the scaffold. In some embodiments, one or more features and/or properties of the scaffold are specified so as to mimic a basement membrane to help to induce the secretory phenotype of mammary epithelial cells in vitro.

### CROSS-REFERENCE

This application claims the benefit of and priority to U.S. Provisional Patent Application No. 63/299,349, filed Jan. 13, 2022, which is hereby incorporated by reference in its entirety for all purposes.

### BACKGROUND

Milk is a staple of the human diet, both during infancy and throughout life. The American Academy of Pediatrics and World Health Organization recommend that infants be exclusively breastfed for the first 6 months of life, and consumption of dairy beyond infancy is a mainstay of human nutrition, representing a 700 billion dollar industry worldwide. However, lactation is a physiologically demanding and metabolically intensive process that can present biological and practical challenges for breastfeeding mothers, and milk production is associated with environmental, social, and animal welfare impacts in agricultural contexts.

The possibility of using mammalian cell culture to produce food has gained increasing interest in recent years, with the development of several successful prototypes of meat and sea food products from cultured muscle and fat cells (Stephens et al. 2018 Trends Food Sci Technol. 78:155-166). Additionally, efforts are underway to commercialize the production of egg and milk proteins using microbial expression systems. However, this fermentation-based process relies on a cellular microenvironment that best mimics in vivo conditions to help proliferate mammalian cell culture.

### SUMMARY OF THE DISCLOSURE

Disclosed herein, in certain embodiments, are cell constructs, comprising: a) a three dimensional scaffold comprising a plurality of fibers that are non-uniformly oriented and/or non-linearly oriented and that comprise thermoplastic polyurethane (TPU) and/or polycaprolactone (PCL), said three dimensional scaffold having an exterior surface, an interior surface defining an interior cavity/basal chamber, said three dimensional scaffold being at least partially permeable from the interior surface to the exterior surface; b) a culture media disposed within the interior cavity/basal chamber and in fluidic contact with the internal surface; and c) an at least partially confluent monolayer of polarized mammary cells coupled to the exterior surface of the three-dimensional scaffold, or a portion thereof, wherein the mammary cells comprise mammary epithelial cells, mammary myoepithelial cells, and/or mammary progenitor cells. In some embodiments, the polarized mammary cells comprise an apical surface and a basal surface. In some embodiments, the basal surface of the mammary cells is in fluidic contact with the culture media. In some embodiments, the three dimensional scaffold is configured to mimic a basement membrane of a mammary gland based on a specified set of one or more features for said three dimensional scaffold. In some embodiments, the one or more features comprise one or more topological features, one or more mechanical properties, one or more surface properties, one or more viscoelastic properties, or a combination thereof. In some embodiments, the one or more topological features comprise i) an average fiber diameter of the plurality of fibers, ii) orientation(s) of the plurality of fibers, or iii) a combination thereof. In some embodiments, the average fiber diameter is from about 5 nm to about 5000 nm, from about 5 nm to about 50 nm, from about 50 nm to about 150 nm, from about 100 nm to about 500 nm, from about 100 nm to about 300 nm, from about 200 nm to about 1000 nm, from about 500 nm to about 1500 nm, from about 1000 nm to about 3000 nm, from about 1500 nm to about 5000 nm, or from about 1000 nm to about 10000 nm. In some embodiments, the orientation(s) of the plurality of fibers correlates to a specified extent of randomness. In some embodiments, the one or more mechanical properties comprises i) a thickness of the three dimensional scaffold, ii) a modulus of elasticity of the three dimensional scaffold, iii) a permeability of the three dimensional scaffold, or iv) a combination thereof. In some embodiments, the thickness of the three dimensional scaffold is from about 20 μm to about 100 μm. In some embodiments, the modulus of elasticity of the three dimensional scaffold is from about 100 Pa to about 300 Pa. In some embodiments, the three dimensional scaffold comprises a plurality of pores extending from the interior surface to the exterior surface, thereby enabling said permeability. In some embodiments, the plurality of pores define corresponding channel(s) that pass through the three dimensional scaffold. In some embodiments, the permeability of the three dimensional scaffold correlates to a porosity of the three dimensional scaffold. In some embodiments, the porosity of the scaffold is from about 5% to about 95%, from about 15% to about 75%, from about 25% to about 70%, or from about 40% to about 60%. In some embodiments, the three dimensional scaffold has a specified density. In some embodiments, the plurality of pores have an average maximum dimension across the exterior surface from about 5 nm to about 1000 nm, from about 5 nm to about 50 nm, from about 50 nm to about 150 nm, from about 100 nm to about 500 nm, or from about 250 nm to about 1000 nm. In some embodiments, the plurality of pores have an average maximum dimension across the exterior surface from about 8 nm to about 10 nm, from about 25 nm to about 75 nm, from about 100 nm to about 250 nm, from about 200 nm to about 400 nm, or from about 300 nm to about 600 nm. In some embodiments, the plurality of pores have an average maximum dimension across the exterior surface that is less than or about the same as the average size (in diameter or length or as measured and/or sorted using a cell strainer giving rise to the average size definition for the cells) of the mammary cells. In some embodiments, the average size of the mammary cells is determined in a non-lactation stage of the cells. In some embodiments, one or more of the fibers comprises one or more polymer chains of a polymer material. In some embodiments, the polymer material comprises thermoplastic polyurethane and/or polycaprolactone. In some embodiments, the one or more viscoelastic properties of the scaffold is based on a degree of entanglement of a polymer chain of the fibers. In some embodiments, the one or more viscoelastic properties of the scaffold is based on a ratio of a degree of entanglement of a polymer chain of the fibers with itself to a degree of entanglement of two or more polymer chains of the fibers. In some embodiments, the degree of entanglement is determined via the Gauss Linking Integral. In some embodiments, the one or more surface properties comprises i) a specific surface area of the three dimensional scaffold, ii) specified hydrophobicity and/or hydrophilicity at specified region(s) of the three dimensional scaffold, iii) a surface charge of the three dimensional scaffold, iv) one or more surface coatings applied to the three dimensional scaffold, v) an extent of the one or more surface coatings, or vi) a combination thereof. In some embodiments, the specific surface area is a specified amount. In some embodiments, the hydrophobicity and/or hydrophilicity of the three dimensional scaffold is based on a surface treatment applied to the three dimensional scaffold. In some embodiments, the surface treatment includes plasma treatment. In some embodiments, the surface charge of the three dimensional scaffold is based on a surface treatment applied to the three dimensional scaffold. In some embodiments, the surface treatment includes poly-l-lysine coating to make surface more positively charged for cell attachment, and/or coating with mussel-inspired adhesive L-DOPA for enhanced cell attachment. In some embodiments, the one or more surface coatings comprise a matrix material. In some embodiments, the matrix material comprises one or more extracellular matrix proteins. In some embodiments, the matrix material comprises Collagen-IV, Laminin-1, RGD peptide, laminin peptides like IKVAV, other ECM-peptides, or a combination thereof. In some embodiments, the extent of the one or more surface coatings corresponds to a specified amount of protein on the exterior surface. In some embodiments, the exterior surface is uncoated. In some embodiments, a population of the plurality of fibers are nanofibers (e.g., fibers having a diameter or thickness in the nanometer range, as described herein). In some embodiments, the fibers further comprise polyether sulfone (PES), polysulfone (PS), and/or polyvinylidene fluoride (PVDF). In some embodiments, the plurality of fibers are hollow. In some embodiments, the plurality of fibers are electrospun, wet spun, dry spun, melt spun, phase inversion spun, or a combination thereof. In some embodiments, the three dimensional scaffold is configured to activate a Jak2-Stat5 milk biosynthetic pathway via the mammary cells. In some embodiments, at least 70%, at least 80%, at least 90%, at least 95%, at least 99%, or 100% of the mammary cells are polarized in the same orientation. In some embodiments, the monolayer of polarized mammary cells is at least 70% confluent, at least 80% confluent, at least 90% confluent, at least 95% confluent, at least 99% confluent, or 100% confluent. In some embodiments, the mammary cells comprise a constitutively active prolactin receptor protein. In some embodiments, the culture medium comprises prolactin. In some embodiments, the three dimensional scaffold comprises a sheet configuration, a mat configuration, a sphere configuration, or a tube configuration. In some embodiments, the tube configuration defines one or more conduits. In some embodiments, the mat configuration is configured to be folded so as to form the tube configuration.

Disclosed herein, in certain embodiments, are methods of producing an isolated cultured milk product from mammary cells, comprising: a) culturing a cell construct in a bioreactor under conditions which produce the cultured milk product, said cell construct comprising: i) a three dimensional scaffold comprising a plurality of fibers that are non-uniformly oriented and/or non-linearly oriented and that comprise thermoplastic polyurethane and/or polycaprolactone, said three dimensional scaffold having an exterior surface, an interior surface defining an interior cavity/basal chamber, said three dimensional scaffold being at least partially permeable from the interior surface to the exterior surface; ii) a culture media disposed within the interior cavity/basal chamber and in fluidic contact with the internal surface; and iii) an at least partially confluent monolayer of polarized mammary cells coupled to the exterior surface of the three-dimensional scaffold, or a portion thereof, wherein the mammary cells comprise mammary epithelial cells, mammary myoepithelial cells, and/or mammary progenitor cells; and b) isolating the cultured milk product. In some embodiments, for any method described herein, the cell construct comprises any cell construct described herein. In some embodiments, the bioreactor comprises an apical compartment that is substantially isolated from the internal cavity of the cell construct. In some embodiments, a basal surface of the mammary cells is in fluidic contact with the culture media. In some embodiments, the apical compartment is in fluidic contact with an apical surface of the mammary cells. In some embodiments, the cultured milk product is secreted from the apical surface of the mammary cells into the apical compartment. In some embodiments, the cell construct further comprises a plurality of plasma cells disposed on the exterior surface. In some embodiments, the cultured milk product comprises secretory IgA (sIgA), IgM (sIgM), and/or IgG. In some embodiments, a total cell density of plasma cells in the bioreactor is about 200 to 500 plasma cells per mm². In some embodiments, the culture media substantially does not contact the cultured milk product. In some embodiments, the total cell density of mammary cells within the bioreactor is at least 10¹¹; and alternatively wherein total surface area of mammary cells within the bioreactor is at least about 450 cm² or about 1.0 m² or about 1.5 m². In some embodiments, the total surface area of mammary cells within the bioreactor is at least about 300 cm², 450 cm², or 500 cm². In some embodiments, the total cell density of mammary cells within the bioreactor is at least about 500 to about 1,500 mammary cells per mm², such as about 600 to about 1,000 mammary cells per mm², about 500 to about 100,000 mammary cells per mm² or about 1000 to about 50,000 mammary cells per mm². In some embodiments, the culturing is carried out at a temperature of about 27° C. to about 39° C. In some embodiments, the culturing is carried out at an atmospheric concentration of CO₂ of about 4% to about 6%.

Disclosed herein, in certain embodiments, are bioreactors, comprising: a) an apical compartment comprising a cultured milk product; and b) at least one live cell construct comprising: i) a three dimensional scaffold comprising a plurality of fibers that are non-uniformly oriented and/or non-linearly oriented and that comprise thermoplastic polyurethane and/or polycaprolactone, said three dimensional scaffold having an exterior surface, an interior surface defining an interior cavity/basal chamber, said three dimensional scaffold being at least partially permeable from the interior surface to the exterior surface; ii) a culture media disposed within the interior cavity/basal chamber and in fluidic contact with the internal surface; and iii) an at least partially confluent monolayer of polarized mammary cells coupled to the exterior surface of the three-dimensional scaffold, or a portion thereof, wherein the mammary cells comprise mammary epithelial cells, mammary myoepithelial cells, and/or mammary progenitor cells. In some embodiments, for any bioreactors described herein, the cell construct comprises any cell construct described herein. In some embodiments, the total cell density of mammary cells within the bioreactor is at least 10¹¹. In some embodiments, the total surface area of mammary cells within any of the bioreactors is at least about 450 cm² or about 1.0 m² or about 1.0 m² or about 1.5m². In some embodiments, the total surface area of mammary cells within the bioreactor is at least about 300 cm², 450 cm², or 500 cm². In some embodiments, the total cell density of mammary cells within any of the bioreactors is at least about 500 to about 1,500 mammary cells per mm², such as about 600 to about 1,000 mammary cells per mm², about 500 to about 100,000 mammary cells per mm² or about 1000 to about 50,000 mammary cells per mm²

Disclosed herein, in certain embodiments, are methods for producing a scaffold for isolated cultured milk production from mammary cells, comprising forming a porous mat comprising a plurality of fibers that are non-uniformly oriented and/or non-linearly oriented, said fibers comprising thermoplastic polyurethane and/or polycaprolactone. In some embodiments, any of the methods further comprises folding the porous mat into a tubular configuration. In some embodiments, forming the porous mat comprises electrospinning, wet spinning, dry spinning, melt spinning, and/or phase inversion spinning of thermoplastic polyurethane and/or polycaprolactone to form a plurality of fibers. In some embodiments, forming the porous mat comprises electrospinning, wet spinning, dry spinning, melt spinning, and/or phase inversion spinning of other polymer material such as polyether sulfone (PES), polysulfone (PS), and/or polyvinylidene fluoride (PVDF) to form a plurality of fibers. In some embodiments, the porous mat comprises an exterior surface, an interior surface defining an interior cavity/basal chamber, and a plurality of pores extending from the interior surface to the exterior surface. In some embodiments, forming the porous mat creates a specified set of one or more features for said scaffold. In some embodiments, the one or more features comprise one or more topological features, one or more mechanical properties, one or more surface properties, one or more viscoelastic properties, or a combination thereof. In some embodiments, the one or more topological features comprise i) an average fiber diameter of the plurality of fibers, ii) orientation(s) of the plurality of fibers, or iii) a combination thereof. In some embodiments, the average fiber diameter is from about 5 nm to about 5000 nm, from about 5 nm to about 50 nm, from about 50 nm to about 150 nm, from about 100 nm to about 500 nm, from about 100 nm to about 300 nm, from about 200 nm to about 1000 nm, from about 500 nm to about 1500 nm, from about 1000 nm to about 3000 nm, or from about 1500 nm to about 5000 nm. In some embodiments, the orientation(s) of the plurality of fibers correlates to a specified extent of randomness. In some embodiments, the one or more mechanical properties comprises i) a thickness of the three dimensional scaffold, ii) a modulus of elasticity of the three dimensional scaffold, iii) a porosity of the three dimensional scaffold, or iv) a combination thereof. In some embodiments, the thickness of the scaffold is from about 20 μm to about 100 μm. In some embodiments, the modulus of elasticity of the scaffold is from about 100 Pa to about 300 Pa. In some embodiments, the porosity of the scaffold is from about 5% to about 95%, from about 15% to about 75%, from about 25% to about 70%, or from about 40% to about 60%. In some embodiments, the scaffold has a specified density. In some embodiments, the plurality of pores have an average maximum dimension across the exterior surface from about 5 nm to about 1000 nm, from about 5 nm to about 50 nm, from about 50 nm to about 150 nm, from about 100 nm to about 500 nm, or from about 250 nm to about 1000 nm. In some embodiments, the plurality of pores have an average maximum dimension across the exterior surface from about 8 nm to about 10 nm, from about 25 nm to about 75 nm, from about 100 nm to about 250 nm, from about 200 nm to about 400 nm, or from about 300 nm to about 600 nm. In some embodiments, one or more of the fibers comprises one or more polymer chains of a polymer material. In some embodiments, the polymer material comprises thermoplastic polyurethane and/or polycaprolactone. In some embodiments, the one or more viscoelastic properties of the scaffold is based on a degree of entanglement of a polymer chain of the fibers. In some embodiments, the one or more viscoelastic properties of the scaffold is based on a ratio of a degree of entanglement of a polymer chain of the fibers with itself to a degree of entanglement of two or more polymer chains of the fibers. In some embodiments, the degree of entanglement is determined via the Gauss Linking Integral. In some embodiments, the one or more surface properties comprises i) a specific surface area of the scaffold, ii) specified hydrophobicity and/or hydrophilicity at specified region(s) of the scaffold, iii) a surface charge of the scaffold, iv) one or more surface coatings applied to the scaffold, v) an extent of the one or more surface coatings, or vi) a combination thereof. In some embodiments, the specific surface area is a specified amount. In some embodiments, the hydrophobicity and/or hydrophilicity of the scaffold is based on a surface treatment applied to the scaffold. In some embodiments, the surface treatment includes plasma treatment. In some embodiments, the surface charge of the scaffold is based on a surface treatment applied to the scaffold. In some embodiments, the surface treatment includes poly-l-lysine coating to make surface more positively charged for cell attachment, and/or coating with mussel-inspired adhesive L-DOPA for enhanced cell attachment. In some embodiments, the one or more surface coatings comprise a matrix material. In some embodiments, the matrix material comprises one or more extracellular matrix proteins. In some embodiments, the matrix material comprises Collagen-IV, Laminin-1, RGD peptide, laminin peptides like IKVAV, other ECM-peptides, or a combination thereof. In some embodiments, the extent of the one or more surface coatings corresponds to a specified amount of protein on the exterior surface. In some embodiments, the exterior surface is uncoated. In some embodiments, a population of the plurality of fibers are nanofibers (e.g., fibers having a diameter or thickness in the nanometer range, as described herein) In some embodiments, the plurality of fibers are hollow.

Disclosed herein, in certain embodiments, are scaffolds for isolated cultured milk production from mammary cells formed by any method described herein.

These and other aspects of the disclosure are set forth in more detail in the description of the disclosure below.

### BRIEF DESCRIPTION OF THE DRAWINGS

FIG. 1 shows an example of the collection of milk for nutritional use from mammary epithelial cells grown as a confluent monolayer in a compartmentalizing culture apparatus in which either fresh or recycled media is provided to the basal compartment and milk is collected from the apical compartment. TEER, transepithelial electrical resistance.

FIG. 2 shows an example of polarized absorption of nutrients and secretion of milk across a confluent monolayer of mammary epithelial cells anchored to a scaffold at the basal surface.

FIG. 3 shows an example micropatterned scaffold that provides increased surface area for the compartmentalized absorption of nutrients and secretion of milk by a confluent monolayer of mammary epithelial cells.

FIG. 4 shows three examples of a hollow fiber bioreactor depicted as a bundle of capillary tubes (top), which can support mammary epithelial cells lining either the external (top and lower left) or internal (lower right) surface of the capillaries, providing directional and compartmentalized absorption of nutrients and secretion of milk.

FIG. 5 exemplifies a cross-section of three-dimensional cell construct. The construct is made up of a scaffold having an interior surface defining an interior cavity/basal chamber and an exterior surface. The interior cavity/basal chamber comprises cell culture media. A matrix material sits on top of the exterior surface of the scaffold. Pores transverse the scaffold from the interior surface to the exterior surface, allowing cell media to contact the basal surface of the cells of the cell monolayer disposed on the matrix material.

FIG. 6 exemplifies a bioreactor for producing a cultured milk product. The bioreactor is made up of a cell construct and an apical chamber. The cell construct is made up of a scaffold having an interior surface defining an interior cavity/basal chamber and an exterior surface. The cavity comprises cell culture media. A matrix material sits on top of the exterior surface of the scaffold. Pores transverse the scaffold from the interior surface to the exterior surface, allowing cell media to contact the basal surface of the cells of the cell monolayer disposed on the matrix material. The apical surface of the cells of the cell monolayer secrete the milk/cultured milk product into the apical chamber. The apical chamber and the interior cavity/basal chamber are separated by the cell monolayer.

FIG. 7 exemplifies a cell construct. The construct is made up of a scaffold having an interior surface defining an interior cavity/basal chamber and an exterior surface. The interior cavity/basal chamber comprises cell.

FIG. 8 exemplifies a cell construct having mammary epithelial cells (MECs) and plasma cells. The plasma cells are adjacent to the scaffold. The MECs form a confluent monolayer above (and in some instances, in between) the plasma cells, with the apical side of the MECs facing the apical compartment (or, milk compartment). The plasma cells secrete IgA, which then binds to a receptor on the basolateral surface of the MECs, triggering internalization of the antibody- receptor complex and further processing of the antibody into sIgA as it transits toward the apical surface (not shown).

FIG. 9A depicts actin staining relating to Lonza mammary epithelial cell growth on scaffolds comprising thermoplastic polyurethane (TPU).

FIG. 9B depicts E-Cadherin staining relating to Lonza mammary epithelial cell growth on scaffolds comprising TPU.

FIG. 10A depicts actin staining relating to Lonza mammary epithelial cell growth on scaffolds comprising polycaprolactone (PCL).

FIG. 10B depicts E-Cadherin staining relating to Lonza mammary epithelial cell growth on scaffolds comprising PCL.

FIG. 11A depicts actin staining relating to Lonza mammary epithelial cell growth on conventional plastic.

FIG. 11B depicts E-Cadherin staining relating to Lonza mammary epithelial cell growth on conventional plastic.

FIG. 12 depicts actin and E-Cadherin staining relating to Sigma mammary epithelial cell growth on scaffolds comprising TPU.

FIG. 13 depicts actin and E-Cadherin staining relating to Sigma mammary epithelial cell growth on scaffolds comprising PCL.

FIG. 14 depicts actin and E-Cadherin staining relating to Sigma mammary epithelial cell growth on conventional plastics.

FIG. 15 depicts hematoxylin and eosin (H&E) staining relating to Lonza mammary epithelial cell growth on scaffolds comprising TPU.

FIG. 16A depicts an assay for Lonza mammary cell growth on scaffolds comprising TPU, PCL, polyvinylidene fluoride (PVDF), and gelatin

FIG. 16B depicts a graph outputting an Alamar Blue Assay based on the assay from FIG. 16A.

FIG. 17A depicts an assay for Sigma mammary cell growth on scaffolds comprising TPU, PCL, polyvinylidene fluoride (PVDF), and gel.

FIG. 17B depicts a graph outputting an Alamar Blue Assay based on the assay from FIG. 17A.

FIG. 18 depicts a fluorescence ratio in cell laden TPU and PCL scaffolds (comprising a plurality of nanofibers) compared to a blank TPU and PCL scaffold (comprising a plurality of nanofibers) control.

FIGS. 19A-19D depict average fiber diameter (FIG. 19A), pore size (FIG. 19B), pore diameter (FIG. 19C), and porosity (FIG. 19D) quantitated from scanning electron microscopy images of scaffolds comprising polycaprolactone (PCL) and thermoplastic polyurethane (TPU). N=10 image replicates. Error bars depict standard deviation. p<0.05 for all figures (All pairs using ANOVA).

FIG. 20A depicts average scaffold thickness taken from scanning electron microscopy cross-section images of scaffolds comprising polycaprolactone (PCL) and thermoplastic polyurethane (TPU). Error bars depict standard deviation. p<0.05 for all figures (All pairs using ANOVA).

FIG. 20B exemplifies scanning electron microscopy images, segmented images, and particle analysis of scaffolds comprising polycaprolactone (PCL) and thermoplastic polyurethane (TPU). N=10 image replicates. Error bars depict standard deviation. p<0.05 for all figures (All pairs using ANOVA).

FIGS. 21A-21E depict 240L-D1 cell density (FIG. 21A), 240L-D1 confluence (FIG. 21B), BMQ hMEC a hMEC cell line cell density (FIG. 21C), and BMQ hMEC confluence (FIG. 21D) quantitated from day 10 DAPI and actin stain images of cell-laden scaffolds comprising polycaprolactone (PCL) and thermoplastic polyurethane (TPU). (FIG. 21E) depicts transepithelial electrical resistance values over 24 days of BMQ hMEC-laden scaffolds comprising of PCL and TPU compared to a commercial PET control. Error bras depict standard deviation.

FIG. 22 exemplifies DAPI and actin staining images used for cell density and confluence quantification related to 240L-D1 mammary epithelial cells on scaffolds comprising polycaprolactone (PCL).

FIG. 23 depicts DAPI, Actin, and E-cadherin staining and an Overlay of all three related to 240L-D1 mammary epithelial cells on tissue culture polystyrene without extracellular matrix coating (TCPS-ECM) control compared to scaffolds comprising polycaprolactone (PCL) with (PCL+ECM) and without (PCL-ECM) the addition of extracellular matrix coating at 10× and 20× magnification.

### DETAILED DESCRIPTION

This disclosure is not intended to be a detailed catalog of all the different ways in which the disclosure may be implemented, or all the features that may be added to the instant disclosure. For example, features illustrated with respect to one embodiment may be incorporated into other embodiments, and features illustrated with respect to a particular embodiment may be deleted from that embodiment. Numerous variations and additions to the various embodiments suggested herein will be apparent to those skilled in the art in light of the instant disclosure, which do not depart from the instant disclosure. Hence, the following specification is intended to illustrate some particular embodiments of the disclosure, and not to exhaustively specify all permutations, combinations, and variations thereof.

Unless the context indicates otherwise, it is specifically intended that the various features of the disclosure described herein can be used in any combination. Moreover, the present disclosure also contemplates that in some embodiments of the disclosure, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

### Definitions

Unless otherwise defined, all technical and scientific terms used herein have the same meaning as commonly understood by one of ordinary skill in the art to which this disclosure belongs. The terminology used in the description of the disclosure herein is for the purpose of describing particular embodiments only and is not intended to be limiting of the disclosure.

The terminology used herein is for the purpose of describing particular embodiments only and is not intended to be limiting. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, elements, and/or components, but do not preclude the presence or addition of one or more other features, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

Moreover, the present disclosure also contemplates that in some embodiments, any feature or combination of features set forth herein can be excluded or omitted.

Furthermore, the term “about,” as used herein when referring to a measurable value such as an amount of a compound or agent of this disclosure, dose, time, temperature, and the like, is meant to encompass variations of ±10%, ±5%, ±1%, ±0.5%, or even ±0.1% of the specified amount.

As used herein, the transitional phrase “consisting essentially of” is to be interpreted as encompassing the recited materials or steps and those that do not materially affect the basic and novel characteristic(s) of the disclosure. Thus, the term “consisting essentially of” as used herein should not be interpreted as equivalent to “comprising.”

As used herein, the compositions described in the present disclosure are referred to interchangeably as (the singular or plural forms of) “nutritional compositions substantially similar to human milk,” “milk products,” “milk compositions,” “cultured milk products,” or equivalent as made clear by the context and mean the product secreted by the apical surface of a live cell construct (or, cell culture) comprising human mammary epithelial cells (hMEC). In some embodiments, the live cell construct is cultured in a bioreactor.

As used herein, the term “nanofiber” refers to fibers having a diameter or thickness in the nanometer range. For example, nanofibers may have a diameters or thicknesses ranging from about 0.1 nm to about 100000 nm, including from about 1 nm to about 1000 nm.

As used herein, by “isolate” (or grammatical equivalents, e.g., “extract”) a product, it is meant that the product is at least partially separated from at least some of the other components in the starting material.

The term “polarized” as used herein in reference to cells and/or monolayers of cells refers to a spatial status of the cell wherein there are two distinct surfaces of the cell, e.g., an apical surface and a basal surface, which may be different. In some embodiments, the distinct surfaces of a polarized cell comprise different surface and/or transmembrane receptors and/or other structures. In some embodiments, individual polarized cells in a continuous monolayer have similarly oriented apical surfaces and basal surfaces. In some embodiments, individual polarized cells in a continuous monolayer have communicative structures between individual cells (e.g., tight junctions) to allow cross communication between individual cells and to create separation (e.g., compartmentalization) of the apical compartment and basal compartment.

As used herein, “apical surface” means the surface of a cell that faces an external environment or toward a cavity or chamber, for example the cavity of an internal organ. With respect to mammary epithelial cells, the apical surface is the surface from which the cultured milk product is secreted.

As used herein, “basal surface” means the surface of a cell that is in contact with a surface, e.g., the matrix of a bioreactor.

As used herein, “bioreactor” means a device or system that supports a biologically active environment that enables the production of a cultured milk product described herein from mammary cells described herein.

The term “lactogenic” as used herein refers to the ability to stimulate production and/or secretion of milk. A gene or protein (e.g., prolactin) may be lactogenic, as may any other natural and/or synthetic product. In some embodiments, a lactogenic culture medium comprises prolactin, thereby stimulating production of milk by cells in contact with the culture medium.

As used herein, the term “food grade” refers to materials considered non-toxic and safe for consumption (e.g., human and/or other animal consumption), e.g., as regulated by standards set by the U.S. Food and Drug Administration.

### Cell Constructs

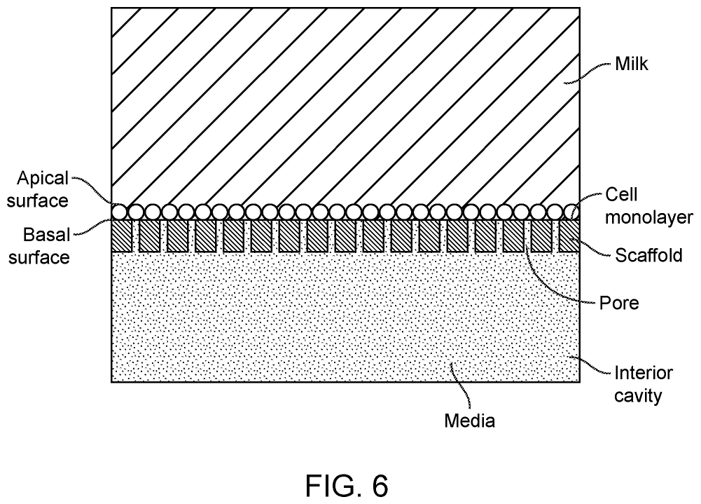

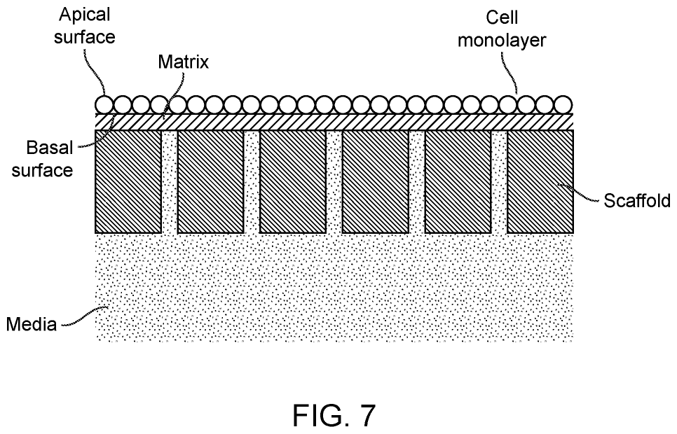

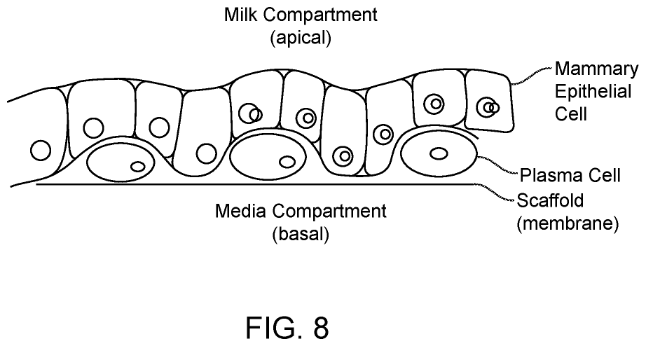

Described herein, in certain embodiments, are cell constructs for producing a cultured milk product from mammary epithelial cells (MECs). In some embodiments, the cell constructs comprise a scaffold, a culture medium in fluidic contact with the scaffold, and mammary cells coupled to the scaffold. In some embodiments, the scaffold comprises a bottom surface/interior surface in fluid contact with the culture medium. In some embodiments, the scaffold comprises a top surface/exterior surface coupled to the MECs. In some embodiments, the MECs are coupled to the exterior surface in a continuous monolayer arrangement. In some embodiments, as described herein, the MECs are polarized and comprise an apical surface, and a basal surface, wherein the basal surface faces towards the exterior surface of the scaffold (see for example FIGS. 6-8).

In some embodiments, the cell constructs enable for compartmentalization between secreted milk from the mammary cells and the culture medium. In some embodiments, the lower surface (interior surface) of the scaffold is adjacent to a basal compartment. In some embodiments, the apical surface of the continuous monolayer (of the MECs) is adjacent to an apical compartment. In some embodiments, the continuous monolayer secretes milk through its apical surface into the apical compartment, thereby producing milk. In some embodiments, the monolayer of mammary cells forms a barrier that divides the apical compartment and the basal compartment, wherein the basal surface of the mammary cells is attached to the scaffold and the apical surface is oriented toward the apical compartment. In some embodiments, the milk product represents the biosynthetic output of cultured mammary epithelial cells (immortalized or from primary tissue samples) and immunoglobin A (IgA), immunoglobin G (IgG), and/or immunoglobin M (IgM) producing cells, for example plasma cells.

### Scaffolds

In some cases, features and/or properties of the scaffold are varied so as to help further the proliferation of mammary epithelial cells. For example, cellular microenvironment plays an important role in driving crucial cellular processes. In the context of mammary epithelial cells, the cellular microenvironment drives processes such as epithelial cell growth, epithelial differentiation and maintenance of epithelial phenotype, polarization, and production and secretion of milk components. The basement membrane (BM), which forms the physical boundary of the mammary gland and provides a support (or scaffolding) for the mammary epithelial cells can impact the development of the mammary gland through its influence on the mammary epithelial cell processes.

Generally, the basement membrane is a thin sheet that physically surrounds the mammary gland and can comprise of cross-linked fibrous networks (for example, comprising a plurality of nanofibers), such as Collagen-IV and laminins (predominantly laminin-1), along with other extracellular matrix (ECM) molecules, such as glycoproteins (like Nidogen) and proteoglycans. The basement membrane can serve as a semi-permeable scaffolding that allows for exchange of nutrients and waste metabolites to and from the mammary gland. Further, it also provides compartmentalization (barrier functionality) between secreted milk components and surrounding stroma and blood circulation. Moreover, the basement membrane can directly influence the ability of mammary epithelial cells to execute milk biosynthesis. For example, the basement membrane can provide mammary epithelial cells with i) bio-physical cues-through mechanical stimuli and its fibrous topographical features, and ii) bio-chemical cues-through its interactions with cells surface receptors called integrins. These bio-physical and bio-chemical cues together can influence the biology of mammary epithelial cells by regulating cell proliferation, epithelial differentiation, spatial organization of luminal and myoepithelial cells, polarization, alveologenesis and ductal morphogenesis, and activation of milk biosynthetic pathways and secretion. In some cases, the basement membrane is constantly being remodeled throughout the development, lactation, and involution of mammary glands to allow it to guide and control epithelial cell behavior. In the context of milk biosynthesis, in some cases, the basement membrane can regulate the Jak2-Stat5 pathway, and hence, prolactin signaling through its interactions with integrin receptors. Similarly, the basement membrane at other organ sites, such as kidney, cornea, and blood vessels, have been shown to have organ-specific topographical features. In certain instances, as a non-limiting example, culturing mammary epithelial cells in or on materials derived from a basement membrane associated in vivo with mammary cells or materials similar to materials derived from a basement membrane associated in vivo with mammary cells (including synthetic materials) promotes key functional aspects of such mammary cells, such as polarization and milk protein synthesis and secretion.

Described herein, in some embodiments, are scaffolds (as part of a cell construct, for example, configured to recapitulate one or more aspects of a basement membrane associated in vivo with mammary cells, and in some cases, the scaffolds are configured to induce the secretory phenotype of mammary epithelial cells in vitro. In some embodiments, such one or more aspects of a basement membrane include, for example, the fiber configuration (e.g., orientation of a plurality of fibers, such as nanofibers), porous nature, and/or other topographical features (e.g., mechanical stiffness and viscoelastic properties). In some embodiments, one or more properties and/or features of a scaffold are specified to at least partially mimic a basement membrane associated in vivo with mammary cells (e.g., a mammary gland). In some embodiments, the scaffold are produced with one or more synthetic materials and/or one or more natural materials (as described herein). In some embodiments, the scaffolds are produced in batch operation, continuous operations, or other processes known in the art for large scale production. In some cases, as a non-limiting example, specifying one or more properties and/or features facilitates batch-to-batch consistencies, scale-up and help reduce costs for large scale manufacturing of cell culturing platforms (in contrast with natural basement membrane derived materials which may pose challenges for such scale-up manufacturing and batch to batch consistencies).

### Optimization of Scaffold Features

In some embodiments, as described herein, the scaffold, as part of a cell construct described herein for example, includes a top surface/exterior surface and a bottom surface/interior surface. In some embodiments, the mammary cells are coupled to the top surface/exterior surface of the scaffold, and the bottom surface/interior surface of the scaffold is in fluid contact with the culture medium. In some embodiments, the scaffold comprises a 2-dimensional surface or a 3-dimensional surface (e.g., a 3-dimensional micropatterned surface, and/or as a cylindrical structure that is assembled into bundles). A non-limiting example of a 2-dimensional surface scaffold is a Transwell® filter.

In some embodiments, the scaffold comprises a three-dimensional surface. Non-limiting examples of a three-dimensional micropatterned surface include a microstructured bioreactor, a decellularized tissue (e.g., a decellularized mammary gland or decellularized plant tissue), micropatterned scaffolds fabricated through casting or three-dimensional printing with biological or biocompatible materials, textured surface.

In some embodiments, the scaffold is a three dimensional scaffold. In some embodiments, the scaffold comprises any shape, such as for example a sheet, sphere, mat, tubular structure or conduits. In some embodiments, the three dimensional scaffold comprises a tube structure or a flat sheet. For example, in some embodiments, the three-dimensional scaffold comprises any structure which has an enclosed hollow interior/central cavity. In some embodiments, the three-dimensional scaffold joins with one or more surfaces to form an enclosed interior chamber/basal compartment. For example, the scaffold can join with one or more walls of a bioreactor to form the interior chamber/basal compartment. In some embodiments, the scaffold is a hollow fiber bioreactor. In some embodiments, the three-dimensional scaffold is a tube in which the central cavity is defined by the interior surface of the scaffold. In some embodiments, the three-dimensional scaffold is a hollow sphere in which the central cavity is defined by the interior surface of the scaffold. In some embodiments, the scaffold comprises a mat configuration, which can be folded into a tube. In some embodiments, the tube has a diameter from about 0.1 mm to about 10 mm. In some embodiments, the tube has a diameter from about 0.5 mm to about 5 mm, from about 1 mm to about 3 mm, from about 1.5 mm to about 2.5 mm.

In some embodiments, a three-dimensional scaffold allows the cells (e.g., mammary epithelial cells and/or plasma cells) to grow or interact with their surroundings in all three dimensions. Unlike two-dimensional environments, in some cases, a three-dimensional cell culture allows cells in vitro to grow in all directions, thereby helping approximate the in vivo mammary environment. Further, the three-dimensional scaffold allows for a larger surface area for culture of the cells and for metabolite and gas exchange, plus it enables necessary compartmentalization—enabling the cultured milk product to be secreted into one compartment, while the cell culture media is contacted with the mammary cells and plasma cells via another compartment.

In some embodiments, the scaffold comprises a plurality of fibers (e.g., fibrous scaffold). In some embodiments, a population of the plurality of fibers are nanofibers (e.g., fibers having a diameter or thickness in the nanometer range, as described herein). In some embodiments, the plurality of fibers comprise one or more polymers (e.g., thermoplastic polyurethane, polycaprolactone, polyether sulfone (PES), polysulfone (PS), and/or polyvinylidene fluoride (PVDF)). In some embodiments, the one or more polymers (for example, of the fibers) comprise one or more polymer chains. In some cases, such materials recapitulate one or more bio-physical cues and/or one or more bio-chemical cues provided by the basement membrane. In some embodiments, the scaffold comprises a natural polymer, a biocompatible synthetic polymer, a synthetic peptide, and/or a composite derived from any combination thereof. In some embodiments, a natural polymer useful with this invention includes, but is not limited to, collagen, chitosan, cellulose, agarose, alginate, gelatin, elastin, heparan sulfate, chondroitin sulfate, keratan sulfate, and/or hyaluronic acid. In some embodiments, a biocompatible synthetic polymer useful with this invention includes, but is not limited to, cellulose, polysulfone, polyvinylidene fluoride, polyethylene co-vinyl acetate, polyvinyl alcohol, sodium polyacrylate, an acrylate polymer, polyethylene glycol, thermoplastic polyurethane (TPU), polycaprolactone (PCL), or a combination thereof. In some embodiments, the scaffold comprises TPU and/or PCL.

In some embodiment, the scaffold comprises a plurality of fibers that are oriented in a non-uniformly and/or non-linearly manner. For example, in some embodiments, the orientation for at least some of the plurality of fibers (e.g., from about 1% to about 99%) is a random orientation (thus non-uniform and/or non-linear with each other). For example in some embodiments, at least 1%, 5%, 10%, 20%, 25%, 33%, 50%, 66%, 75%, 80%, 90%, 99%, of the plurality of fibers in the scaffold are in a non-uniform and/or non-linear orientation (as compared with each other).

In some embodiments, the plurality of fibers form a fibrous/filamentous mesh. As described herein, in some embodiments, the plurality of fibers of the scaffold comprise nanofibers. In some embodiments, the fibrous scaffolds (e.g., scaffolds comprising a plurality of fibers, as described herein) are synthetic and can be formed via electrospinning, wet spinning, dry spinning, melt spinning, and/or phase inversion spinning of thermoplastic polyurethane and/or polycaprolactone. In some embodiments, the fibrous scaffolds can further be formed by electrospinning, wet spinning, dry spinning, melt spinning, and/or phase inversion spinning of other polymer material such as polyether sulfone (PES), polysulfone (PS), and/or polyvinylidene fluoride (PVDF). In some embodiments, such synthetic fibrous scaffolds (such as electrospun fibrous scaffolds) allow for tunability with respect to topographical properties and other mechanical properties, as well as surface chemistries. In some embodiments, the scaffold is produced by electrospinning cellulose nanofibers and/or a cylindrical structure that can be assembled into bundles (e.g., a hollow fiber bioreactor).

In some embodiments, the scaffold is at least partially permeable from the interior surface of the scaffold to exterior surface of the scaffold (and/or vice versa). In some embodiments, such permeability allows for fluid communication between the culture medium and the mammary cells coupled to the exterior surface of the scaffold. For example, in some embodiments, such permeability allows for i) the passage of nutrients to the cells, ii) waste to be carried away (e.g., from the cell layer to the culture medium (e.g., cell media), iii) provision of desired products to the cells (such as growth factors), iv) removal of desired products from the cells, v) exclusion of certain factors that may be present from reaching the cells, vi) other transfer of substances between the cell layer and culture media, or vii) any combination thereof.

In some embodiments, the scaffold is porous so as to enable such permeability between the interior surface and the exterior surface. In some embodiments, the scaffold comprises one or more pores (e.g., pores in the fiber walls of the scaffold) that may extend from the interior surface to the exterior surface. For example, in some embodiments, the pores are due to the fibrous configuration of the scaffold, such as due to the alignment and/or orientation of the plurality of fibers of the scaffold. Accordingly, in some embodiments, the one or more pores provides corresponding passageways through the plurality of fibers that allow the culture medium (cell media) to contact the cell layer coupled to the exterior surface of the scaffold (e.g., the basal surface of the cells of the cell monolayer of the MECs, as described herein). In some embodiments, the pore size of the fiber walls (of the scaffold) are specified so as to modify which components will pass through the walls.

In some embodiments, the pore size of a pore on the scaffold refers to a maximum dimension of a cross-section of a pore across the exterior surface of the scaffold. For example, if one of the pores comprises a circular cross-section as it traverses through the scaffold (e.g., from the exterior surface to the interior surface), the pore size refers to the diameter of the circular cross-section (in this case, the maximum dimension) at the exterior surface of the scaffold. In some embodiments, the pore size of a pore is substantially consistent with the maximum dimension of the pore as it traverses through the scaffold from the exterior surface to the interior surface. In some embodiments, the maximum dimension of the pore varies as it traverses through the scaffold from the exterior surface to the interior surface.

In some embodiments, the average diameter of the nanofiber is from about 100 nm to about 600 nm, from about 200 nm to about 500 nm, or from about 300 nm to about 400 nm. In some embodiments, the nanofiber is a flat sheet and has a fiber diameter from about 100 nm to about 600 nm. In some embodiments, the nanofiber is a tube and has a fiber diameter from about 100 nm to about 600 nm. In some embodiments, average fiber diameter for a PCL tube scaffold is higher than for a PCL flat sheet or a TPU flat sheet.

In some embodiments, the porosity of the scaffold is from about 5% to about 95%, from about 15% to about 75%, from about 25% to about 70%, or from about 40% to about 60%. In some embodiments, the porosity of the scaffold is from about 5% to about 95%, from about 15% to about 75%, from about 25% to about 70%, or from about 40% to about 60%. In some embodiments, the porosity of the nanofiber is from about 10% to about 35%, from about 15% to about 30%, or from about 20% to about 25%. In some embodiments, the nanofiber is a flat sheet and has a porosity from about 10% to about 35%. In some embodiments, the nanofiber is a tube and has a porosity from about 10% to about 35%.

In some embodiments, the scaffold has a specified density. In some embodiments, the plurality of pores have an average maximum dimension across the exterior surface from about 5 nm to about 1000 nm, from about 5 nm to about 50 nm, from about 50 nm to about 150 nm, from about 100 nm to about 500 nm, or from about 250 nm to about 1000 nm. In some embodiments, the plurality of pores have an average maximum dimension across the exterior surface from about 8 nm to about 10 nm, from about 25 nm to about 75 nm, from about 100 nm to about 250 nm, from about 200 nm to about 400 nm, or from about 300 nm to about 600 nm. In some embodiments, the plurality of pores have an average maximum dimension across the exterior surface that is less than or about the same as the average size (in diameter or length or as measured and/or sorted using a cell strainer giving rise to the average size definition for the cells) of the mammary cells. In some embodiments, the average size of the mammary cells is determined in a non-lactation stage of the cells.

In some embodiments, the average pore size of the scaffold is from about 1 nanometer 2 (nm²) to about 5 micrometer2 (μm²). In some embodiments, the average pore size of the scaffold is from about 1 nm² to about 20 nm². In some embodiments, the average pore size of the scaffold is from about 5 nm² to about 15 nm². In some embodiments, the average pore size of the scaffold is from about 8 nm² to about 10 nm². In some embodiments, the average pore size of the scaffold is at least about 5 nm². In some embodiments, the average pore size of the scaffold is at least about 9 nm². In some embodiments, the average pore size of the scaffold is at least about 25 nm². In some embodiments, the average pore size of the scaffold is at least about 50 nm². In some embodiments, the average pore size of the scaffold is at least about 100 nm². In some embodiments, the average pore size of the scaffold is at least about 0.5 μm². In some embodiments, the average pore size of the scaffold is at least about 1.0 μm². In some embodiments, the average pore size of the scaffold is at least about 1.5 μm². In some embodiments, the average pore size of the scaffold is at least about 2.0 μm². In some embodiments, the average pore size of the scaffold is at least about 2.5 μm². In some embodiments, the average pore size of the scaffold is at least about 3.0 μm².

In some embodiments, the average pore size of the nanofiber (measured as area, μm²) is from about 5 nm² to about 600 nm², from about 100 nm² to about 500 nm², or from about 300 nm² to about 400 nm². In some embodiments, the nanofiber is a flat sheet and has a fiber pore size from about 5 nm² to about 600 nm². In some embodiments, the nanofiber is a tube and has a fiber pore size from about 100 nm² to about 600 nm². In some embodiments, the pore size for a PCL tube and TPU flat sheet is comparable.

In some embodiments, the average minimum Feret pore diameter of the nanofiber is from about 10 nm to about 600 nm, from about 200 nm to about 500 nm, or from about 300 nm to about 400 nm. In some embodiments, the nanofiber is a flat sheet and has a minimum Feret pore diameter from about 100 nm to about 600 nm. In some embodiments, the nanofiber is a tube and has a minimum Feret pore diameter from about 100 nm to about 600 nm.

In some embodiments, the average Maximum Feret pore diameter of the nanofiber is from about 30 nm to about 1300 nm, from about 200 nm to about 1200 nm, or from about 300 nm to about 1000 nm. In some embodiments, the nanofiber is a flat sheet and has a Maximum Feret pore diameter from about 300 nm to about 1200 nm. In some embodiments, the nanofiber is a tube and has a Maximum Feret pore diameter from about 100 nm to about 1300 nm.

In some embodiments, the average pore size of the scaffold is correlated with a size of protein passing through the scaffold. In some embodiments, the size of protein is correlated with the molecular weight of the protein. In some embodiments, the size of protein is measured in kilodalton (kDa) for example. Accordingly, in embodiments, the size of the protein (e.g., in kDa) that can pass through the pores is measured so as to determine an average pore size of the scaffold.

In some embodiments, the pore size is specified. As described herein, in some embodiments, the pore size is designed to allow the passage of nutrients to the cells, carry away waste, provide desired products to the cells (such as growth factors), to remove desired products from the cells, and/or exclude certain factors that may be present from reaching the cells.

Accordingly, the pore size of the fiber walls can be varied to modify which components will pass through the walls. For example, in some cases, pore size can allow the passage of large proteinaceous molecules, including growth factors, including, but not limited to, epidermal growth factor and platelet-derived growth factor. The person of ordinary skill in the art would understand how to vary the pore size depending upon the components that it is desirable to pass through the fiber walls to reach the cells or to carry material from the cells. As described herein, the pore size for both the scaffold (fiber walls) and/or the matrix material can be varied to allow for such transfer of materials between the cells and culture medium.

As described herein, in some embodiments, the scaffold is formed with one or more specified features configured to mimic that of a basement membrane (for example, a basement membrane associated in vivo with mammary cells). In some embodiments, the one or more specified features comprise one or more topological features, one or more mechanical properties, one or more surface properties, one or more viscoelastic properties, or a combination thereof.

In some embodiments, the one or more topological features of the scaffold are selected from i) an average fiber diameter of the plurality of fibers and ii) orientation(s) of the plurality of fibers. In some embodiments, as described herein, said average fiber diameter and/or orientation of the plurality of the fibers are varied and specified so as to configure the scaffold to at least partially mimic that of a basement membrane (for example, of a mammary gland).

In some embodiments, the average fiber diameter is from about 3 nm to about 10000 nm, from about 5 nm to about 5000 nm, from about 5 nm to about 50 nm, from about 50 nm to about 150 nm, from about 100 nm to about 300 nm, from about 100 nm to about 500 nm, from about 200 nm to about 1000 nm, from about 500 nm to about 1500 nm, from about 1000 nm to about 3000 nm, or from about 1500 nm to about 5000 nm. In some embodiments, the average diameter of the fibers of the plurality of fibers is characterized via SEM imaging.

As described herein, in some embodiments, the plurality of fibers are configured in a non-linear and/or non-uniform orientation. In some embodiments, the orientation of the plurality of fibers are randomly oriented with respect to each other. In some embodiments, the extent of fiber randomness is characterized using a scanning electron microscope (SEM) imaging through fast Fourier transform (FFT). For example, FFT may generate a point cloud from an image, wherein the proximity of points to each other indicates a similarity in orientation. Accordingly, a completely randomized SEM image may generate a homogenous point cloud (no discernable shape), whereas a more oriented sample may generate a skewed point cloud.

In some embodiments, the one or more mechanical properties of the scaffold are selected from: i) a thickness of the scaffold, ii) a modulus of elasticity of the scaffold (e.g., fibers), and iii) porosity (as described herein). In some embodiments, as described herein, said thickness of the scaffold, a modulus of elasticity of the scaffold (e.g., fibers), and/or porosity are varied and specified so as to configure the scaffold to at least partially mimic that of a basement membrane (for example, of a mammary gland).

In some embodiments, the thickness of the scaffold (e.g., comprising the plurality of fibers) is characterized through SEM imaging. In some embodiments, the thickness of the scaffold is from about 10 μm to about 500 μm. In some embodiments, the thickness of the scaffold is from about 15 μm to about 300 μm. In some embodiments, the thickness of the scaffold is from about 20 μm to about 200 μm. In some embodiments, the thickness of the scaffold is from about 20 μm to about 100 μm. In some embodiments, the thickness of the scaffold is from about 25 μm to about 75 μm. In some embodiments, the thickness of the scaffold is at least about 5 μm, 10 μm, 15 μm, or 20 μm. In some embodiments, the thickness of the scaffold is at most about 50 μm, 100 μm, 250 μm, 500 μm, or 1000 μm. In some embodiments, the average thickness of the scaffold is from about 40 nm to about 350 nm, from about 100 nm to about 300 nm, or from about 150 nm to about 200 nm. In some embodiments, the nanofiber is a flat sheet and has an average thickness of the scaffold from about 40 nm to about 150 nm. In some embodiments, the nanofiber is a tube and has an average thickness of the scaffold from about 100 nm to about 350 nm. In some embodiments, the average thickness of a PCL tube is higher than the average thickness of a PCL flat sheet or a TPU flat sheet.

In some embodiments, the modulus of elasticity is characterized through uniaxial tensile testing. In some embodiments, the scaffold comprises a modulus of elasticity from about 50 Pa to about 500 Pa. In some embodiments, the scaffold comprises a modulus of elasticity from about 100 Pa to about 300 Pa. In some embodiments, the scaffold comprises a modulus of elasticity from about 150 Pa to about 200 Pa. In some embodiments, one or more mechanical properties, or other topographical features of the scaffold is characterized using field emission scanning electron microscopy (FESEM).

In some embodiments, the one or more viscoelastic properties correlates to the entanglement of one or more fibers of the scaffold. As used here, “entanglement” means the interaction either i) of a polymer chain with itself (for example, similar to a single string having knots or tangled points with itself), or ii) between multiple polymer chains (for example, similar to multiple strings crossing over one another and forming one or more knots). In some embodiments, the one or more viscoelastic properties of the scaffold is controlled based on a specified ratio of a degree of entanglement of a polymer chain around itself (of a given nanofiber) to a degree of entanglement between two or more polymer chains (of the nanofibers).

In some embodiments, the porosity refers to i) a percent (%) porosity of the scaffold, ii) pore diameter or pore size (as described herein) through nitrogen porosimetry or mercury intrusion pore size analyzers such as Anton PaarMaster or MicroActive AutoPore V 9600, iii) a percent (%) range of porous area characterized through SEM imaging, and/or iv) a range of kD through dextran diffusion assay. In some embodiments, the porosity of the scaffold is correlated with the density of the scaffold, wherein a higher density (of the scaffold materials) correlates with a lower porosity. In some embodiments, the density of the scaffold is measured via a gas pycnometer.

In some embodiments, the one or more surface properties of the scaffold are selected from: i) the specific surface area, ii) hydrophobicity and/or hydrophilicity, iii) surface treatments to alters surface properties of the scaffold, iv) surface coatings, and v) an extent of surface coatings. In some embodiments, as described herein, said the specific surface area, hydrophobicity and/or hydrophilicity, surface treatments to alters surface properties of the scaffold, surface coatings, and/or an extent of surface coatings are varied and specified so as to configure the scaffold to at least partially mimic that of a basement membrane (for example, of a mammary gland).

The specific surface area can be characterized through the Brunauer Emmett Teller (BET) method or through SEM imaging. In some embodiments, the scaffold includes a specific area or region that is hydrophobic and/or a specific area or region that is hydrophilic. In some embodiments, an extent of hydrophobicity and/or hydrophilicity is measured via contact angle measurement. In some embodiments, the scaffold is subject to surface treatments, such as through plasma treatment, so as to alter hydrophobicity and/or hydrophilicity of the scaffold. In some embodiments the scaffold is subject to surface treatments such as poly-l-lysine coating to alter the surface charge (e.g., to make the surface more positively charged for cell attachment). In some embodiments the scaffold is subject to surface treatments such as coating with mussel inspired adhesive L-3,4-dihydroxyphenylalanine (L-DOPA) to alter the surface charge for enhanced cell attachment.

In some embodiments, a surface coating comprises extracellular matrix (ECM) and/or peptide coatings, as described herein for the matrix material (e.g., Collagen-IV, Laminin-1, RGD peptide, laminin peptides like IKVAV, other ECM-peptides). In some embodiments, an extent of a surface coating is varied, such as by specifying a concentration of coating solution, or through characterizing the total protein on the coated scaffold surface. In some embodiments, relative fluorescence units is used if using targeted staining methods for determining ECM coating on the scaffold surface.

### Mammary Cells

In some embodiments, the mammary cells (for example, as part of a cell construct described herein) comprise milk-producing mammary epithelial cells (MECs), contractile myoepithelial cells, and/or progenitor cells that can give rise to both mammary epithelial cells (MECs) and mammary contractile myoepithelial cells. Mammary epithelial cells (MECs) are the only cells that produce milk. In some embodiments, the mammary cells comprise mammary epithelial cells (MECs), primary mammary epithelial cells, mammary myoepithelial cells and mammary progenitor cells. In some embodiments, the mammary cells are obtained from a tissue biopsy of a mammary gland.

In some embodiments, the mammary cells are derived from breast milk-derived stem cells or breast stem cells originating from tissue biopsy of a mammary gland. The epithelial component of breast milk includes not only mature epithelial cells, but also their precursors and stem cells in culture. A subpopulation of breast milk-derived stem cells displays very high multilineage potential, resembling those typical for human embryonic stem cells (hESCs). Breast stem cells may also originate from tissue biopsy of the mammary gland, and include terminally differentiated MECs. Both breast milk-derived stem cells and breast stem cells originating from tissue biopsy of the mammary gland are multi-potent cells that can give rise to MECs or myoepithelial cells.

In some embodiments, at least 50% of the mammary cells of the cells culture are polarized. In some embodiments, at least 55% of the mammary cells of the cell culture are polarized. In some embodiments, at least 60% of the mammary cells of the cell culture are polarized. In some embodiments, at least 65% of the mammary cells of the cell culture are polarized. In some embodiments, at least 70% of the mammary cells of the cell culture are polarized. In some embodiments, at least 75% of the mammary cells of the cell culture are polarized. In some embodiments, at least 80% of the mammary cells of the cell culture are polarized. In some embodiments, at least 85% of the mammary cells of the cell culture are polarized. In some embodiments, at least 90% of the mammary cells of the cell culture are polarized. In some embodiments, at least 95% of the mammary cells of the cell culture are polarized. In some embodiments, at least 100% of the mammary cells of the cell culture are polarized. In some embodiments, substantially all of the mammary cells of the cell construct are polarized (i.e., have an apical surface and a basal surface). In some embodiments, substantially all the mammary cells of the cell construct are polarized and substantially all the polarized cells are oriented in the same direction. For example, in some embodiments, substantially all of the mammary cells have an apical surface and a basal surface, wherein the apical surface of substantially all of the cells is oriented in the same direction and the basal surface of substantially all of the cells is oriented in the same direction.

In some embodiments, the continuous monolayer of mammary cells has at least 50% confluence over the scaffold. In some embodiments, the continuous monolayer of mammary cells has at least 60% confluence over the scaffold. In some embodiments, the continuous monolayer of mammary cells has at least 70% confluence over the scaffold. In some embodiments, the continuous monolayer of mammary cells has at least about 75% confluence over the scaffold. In some embodiments, the continuous monolayer of mammary cells has at least about 80% confluence over the scaffold. In some embodiments, the continuous monolayer of mammary cells has at least about 85% confluence over the scaffold. In some embodiments, the continuous monolayer of mammary cells has at least about 90% confluence over the scaffold. In some embodiments, the continuous monolayer of mammary cells has at least about 95% confluence over the scaffold. In some embodiments, the continuous monolayer of mammary cells has at least about 99% confluence over the scaffold. In some embodiments, the continuous monolayer of mammary cells has 100% confluence over the scaffold.

In some embodiments, the cell density of the continuous monolayer of mammary cells has at least 5.000 cells/cm² on the scaffold. In some embodiments, the cell density of the continuous monolayer of mammary cells has at least 10.000 cells/cm² on the scaffold. In some embodiments, the cell density of the continuous monolayer of mammary cells has at least 20.000 cells/cm² on the scaffold. In some embodiments, the cell density of the continuous monolayer of mammary cells has at least 30.000 cells/cm² on the scaffold. In some embodiments, the cell density of the continuous monolayer of mammary cells has at least 40.000 cells/cm² on the scaffold. In some embodiments, the cell density of the continuous monolayer of mammary cells has at least 50.000 cells/cm² on the scaffold. In some embodiments, the cell density of the continuous monolayer of mammary cells has at least 60.000 cells/cm² on the scaffold. In some embodiments, the cell density of the continuous monolayer of mammary cells has at least 70.000 cells/cm² on the scaffold. In some embodiments, the cell density of the continuous monolayer of mammary cells has at least 80.000 cells/cm² on the scaffold. In some embodiments, the cell density of the continuous monolayer of mammary cells has at least 90.000 cells/cm² on the scaffold. In some embodiments, the cell density of the continuous monolayer of mammary cells has at least 100.000 cells/cm² on the scaffold. In some embodiments, the cell density of the continuous monolayer of mammary cells has at least 150.000 cells/cm² on the scaffold. In some embodiments, the cell density of the continuous monolayer of mammary cells has at least 200.000 cells/cm² on the scaffold.

In some embodiments, the scaffold, or at least portion of the scaffold, is uncoated.

In some embodiments, the top surface/exterior surface of the scaffold is coated with a matrix material. In some embodiments, the matrix is made up of one or more extracellular matrix proteins. Non-limiting examples of extracellular matrix proteins include collagen, laminin, entactin, tenascin, and/or fibronectin. In some embodiments, the top of the scaffold is coated with Laminin-1, Collagen-IV, RGD peptide, laminin peptides like IKVAV, other ECM-peptides, or a combination thereof.

In some embodiments, the matrix material is located between the exterior surface of the scaffold and the mammary epithelial cells. In some embodiments, the matrix material is porous.

In some embodiments, the matrix material is permeable to the cell media, allowing the cell media to contact the cells of the layer of the mammary cells. In some embodiments, the matrix material is transversed by at least one pore that allows the cell media to contact the layer(s) of mammary epithelial cells. In some embodiments, the matrix material comprises pores having an average pore size (as described herein, for example with reference to the scaffold pores) that corresponds with the average pore size of the scaffold (as described herein). In some embodiments, the pores of the matrix material are at least partially aligned with the pores of the scaffold. In some embodiments, the pores of the matrix material are randomly situated, and thereby may or may not be aligned with any of the pores the scaffold. In some embodiments, a ECM-coated PCL scaffold supports the self-organization of cells into distinct structures to a higher extend than uncoated PCL or ECM-coated TPU scaffold.

In some embodiments, the range of the average pore size (as described herein, for example with reference to the scaffold pores) of the pores in the matrix material is similar to the range in the average pore size of the pores for the scaffold, as described herein.

### Genetic Modifications to Mammary Cells

In some embodiments, the mammary cells comprise one or more genetic modification. For example, in some embodiments, the mammary cells comprise a constitutively active prolactin receptor protein. In some embodiments, the mammary cells comprise a constitutively active human prolactin receptor protein. Where the primary mammary epithelial cell or immortalized mammary epithelial cells comprise a constitutively active prolactin receptor, the culture medium does not contain prolactin.

In some embodiments, the constitutively active human prolactin receptor protein comprises a deletion of amino acids, as described in PCT Publication WO2021242866A1, which is incorporated herein in its entirety.

In some embodiments, the mammary cells comprise a loss of function mutation introduced into a circadian related gene PER2, as described in PCT Publication WO2021242866A1, which is incorporated herein in its entirety. In some embodiments, the loss of function mutation introduced into a circadian related gene PER2 promotes increased synthesis of cultured milk components.

In some embodiments, the mammary cells comprise a polynucleotide encoding a prolactin receptor comprising a modified intracellular signaling domain, as described in PCT Publication WO2021242866A1, which is incorporated herein in its entirety. In some embodiments, the loss of function mutation introduced into a circadian related gene PER2 promotes increased synthesis of individual cultured milk components.

In some embodiments, the mammary cells comprise a polynucleotide encoding a modified (e.g., recombinant) effector of a prolactin protein, as described in PCT Publication WO2021242866A1, which is incorporated herein in its entirety. In some embodiments, the modified effector of the prolactin protein comprises a janus kinase-2 (JAK2) tyrosine kinase domain. In some embodiments, the modified effector comprises a JAK2 tyrosine kinase domain fused to a signal transducer and activator of transcription-5 (STAT5) tyrosine kinase domain (e.g., a polynucleotide encoding a JAK2 tyrosine kinase domain linked to the 3′ end of a polynucleotide encoding the STAT5 tyrosine kinase domain). In some embodiments, the modified effector of a prolactin protein promotes increased synthesis of individual cultured milk components.

### Plasma Cells

Plasma cells are derived from a human donor. In some embodiments, the plasma cells are derived from bone marrow, spleen, and/or a lymph node. a primary mammary tissue sample. In certain embodiments, the plasma cells are derived from mucosal epithelial cells other than mammary cells (e.g., from oronasal, gastrointestinal, or respiratory tissue). In some embodiments, the plasma cells are derived from a plasma cell line. In certain embodiments, the plasma cells are derived from a plasmacyte cell line. In some embodiments, the plasma cells are isolated and sorted from non-plasma cells via fluorescence-activated cell sorting, magnetic-activated cell sorting, and/or microfluidic cell sorting. In some embodiments, plasma cells, plasmablasts, or pre-plasmablasts are sorted and isolated by FACS analysis using markers known in the art (e.g., CD38, CD138 and/or CD19). In certain embodiments, the plasma cells are cultivated with the immortalized mammary epithelial cells on a scaffold, thereby producing a cell construct for producing a cultured milk product with secretory products of the plasma cells and mammary cells (e.g., slgA, IgG, and/or sIgM). In certain embodiments, the plasma cells are grown on a scaffold below a monolayer of mammary cells. In certain embodiments, the plasma cells are grown as dispersed populations of plasma cells overlayed by a monolayer of mammary cells. In certain embodiments, the plasma cells are stimulated to produce immunoglobins during co-culture with mammary cells. In certain embodiments, the plasma cells produce one or more immunoglobins of a class selected from IgG, IgM and IgA. In certain embodiments the plasma cells produce IgA and/or IgM. In certain embodiments, plasma cells produce IgA and/or IgM, and the IgA and/or IgM is processed by mammary epithelial cells to yield sIgA and/or sIgM that is bound to secretory component, and the sIgA and/or sIgM is secreted by the apical surface of the mammary cells.

### Bioreactor

Disclosed herein, in certain embodiments, are bioreactors, comprising: (a) an apical compartment comprising a cultured milk product; and (b) at least one cell construct comprising: i) a three dimensional scaffold comprising a plurality of fibers that are non-uniformly oriented and/or non-linearly oriented and that comprise thermoplastic polyurethane and/or polycaprolactone, said three dimensional scaffold having an exterior surface, an interior surface defining an interior cavity/basal chamber, said there dimensional scaffold being at least partially permeable from the interior surface to the exterior surface; ii) a culture media disposed within the interior cavity/basal chamber and in fluidic contact with the internal surface; and iii) an at least partially confluent monolayer of polarized mammary cells coupled to the exterior surface of the three-dimensional scaffold, or a portion thereof, wherein the mammary cells comprise mammary epithelial cells, mammary myoepithelial cells, and/or mammary progenitor cells.; wherein an apical surface of the mammary cells is in fluidic contact with the apical compartment.

In some embodiments, the bioreactor is an enclosed bioreactor. In some embodiments, the apical chamber is substantially isolated from the interior cavity/basal compartment.

A hollow fiber bioreactor is an exemplary bioreactor for use with the methods disclosed here. The hollow fiber bioreactor is a high-density, continuous perfusion culture system that closely approximates the environment in which cells grow in vivo. It consists of thousands of semi-permeable three-dimensional scaffolds (e.g.,, hollow tubes made up of a plurality of fibers, such as electrospun fibers), as described herein, in a parallel array within a cartridge shell fitted with inlet and outlet ports. These fiber bundles are potted or sealed at each end so that any liquid entering the ends of the cartridge will necessarily flow through the interior of the fibers. Cells may be seeded inside and/or outside the fibers within the cartridge in the extra capillary space (ECS). In some embodiments, the hollow fiber bioreactor comprises a single tube made up of a plurality of fibers (e.g., electrospun fibers). In some embodiments, the hollow fiber bioreactor comprises one or more tubes made up of a plurality of fibers.

Three fundamental characteristics differentiate hollow fiber cell culture from other methods: (1) cells are bound to a porous matrix much as they are in vivo, not a plastic dish (for example), (2) the molecular weight cut off of the support matrix can be controlled, and (3) extremely high surface area to volume ratio (150 cm² or more per mL) which provides a large area for metabolite and gas exchange for efficient growth of host cells.

The bioreactor structure includes a fiber matrix (e.g., three-dimensional scaffold as described herein) that allows permeation of nutrients, gases and other basic media components, as well as cell waste products, but not cells, where the cells can be amplified. The hollow fibers help to create a semi-permeable barrier between the cell growth chamber and the medium flow. Since the surface area provided by this design is large, using this fiber as a culture substrate allows the production of large numbers of cells. Cells growing in the 3-dimensional environment within the bioreactor are bathed in fresh medium as it perfuses through the hollow fibers.

In configuring the hollow fiber bioreactor, design considerations and parameters for the scaffold can be varied (as described herein), depending upon the goals associated with expansion of the cells.

### Methods of Making Cell Constructs

Disclosed herein, in certain embodiments, are methods of making a cell construct for producing a cultured milk product. In some embodiments, the cultured milk product comprises immunoglobulins. In some embodiments, the method comprises (a) depositing isolated mammary epithelial cells, mammary myoepithelial cells and/or mammary progenitor cells on the upper surface (exterior surface) of a scaffold having an upper surface and lower surface; (b) cultivating the mammary cells of (a) on the scaffold, to produce a monolayer of polarized mammary cells located above the upper surface of the scaffold, wherein the upper surface is located adjacent to and above the lower surface of the scaffold, and wherein the polarized mammary cells comprise an apical surface and a basal surface, thereby producing a cell construct for producing the cultured milk product. In some embodiments, the mammary cells are primary mammary cells. In some embodiments, the mammary cells are immortalized. In some embodiments, the mammary cells are derived from a cell culture. In some embodiments, the mammary epithelial cells, myoepithelial cells and/or mammary progenitor cells are isolated from bone marrow, spleen tissue, lymph node tissue, mammary explants from mammary tissue (e.g., breast, udder, teat tissue), or raw breastmilk. In some embodiments, the mammary cells comprise mammary epithelial cells. In some embodiments, the mammary cells, comprise mammary myoepithelial cells. In some embodiments, the mammary cells, comprise mammary progenitor cells. In some embodiments, plasma cells are also deposited on the exterior surface of the scaffold, to produce a mixed population of plasma cells and mammary cells (i.e., mammary epithelial cells, mammary myoepithelial cells and/or mammary progenitor cells). In some embodiments, one or more properties and features of the scaffold is specified (as described herein) so as to help mimic a basement membrane. In some embodiments, the plasma cells are deposited onto the surface of the scaffold prior to the deposition of the mammary cells. In some embodiments, the plasma cells are isolated from any suitable human tissue or a cell culture.

In certain embodiments, the plasma cells are stimulated to produce immunoglobins during co-culture. In certain embodiments, the plasma cells produce one or more immunoglobins of a class selected from IgG, IgM and IgA. In certain embodiments the plasma cells produce secretory IgA. In certain embodiments, plasma cells are co-cultured with MECs in a bioreactor according to methods described herein. In certain embodiments, the bioreactor is a hollow fiber bioreactor described herein.

In certain embodiments, mammary cells are modified and/or stimulated with prolactin according to the methods described herein to stimulate and optimize milk production. In certain embodiments, the mammary cells are modified to express a constitutively active prolactin receptor protein.

In certain embodiments, mammary cells are identified and isolated from mammary tissue samples. In some embodiments, the mammary cells are isolated and sorted via fluorescence-activated cell sorting, magnetic-activated cell sorting, and/or microfluidic cell sorting. In certain embodiments, the mammary epithelial cell populations are sorted by FACS analysis using markers known in the art for identifying the cell populations. In certain embodiments, myoepithelial mammary cells and luminal epithelial mammary cells are isolated by FACS analysis. In certain embodiments, progenitor myoepithelial mammary cells and/or progenitor luminal epithelial mammary cells are isolated by FACS analysis. Any suitable method known in the art for sorting mammary epithelial cells (e.g., luminal epithelial cells), myoepithelial cells, progenitor cells, and immune cells can be used. For example, mammary cells can be sorted using CD24, EPCAM and/or CD49f, cell surface markers.

In some embodiments, plasma cells are identified and isolated from primary mucosal tissue (e.g., oronasal, gastrointestinal, respiratory or mammary). In some embodiments, plasma cells are identified and isolated from primary mammary tissue samples. In some embodiments, the plasma cells are isolated and sorted via fluorescence-activated cell sorting, magnetic-activated cell sorting, and/or microfluidic cell sorting. In certain embodiments, plasma cells are sorted and isolated by FACS analysis. In certain embodiments plasma cells, plasmablasts, or pre-plasmablasts are sorted and isolated by FACS analysis using markers known in the art (e.g., CD20, CD38, CD138 and/or CD19).

In some embodiments, the culturing and/or cultivating of the mammary cells and/or plasma cells for the cell construct is carried out at a temperature of about 35° C. to about 39° C. (e.g., a temperature of about 35° C., 35.5° C., 36° C., 36.5° C., 37° C., 37.5° C., 38° C., 38.5° C. or about 39° C., or any value or range therein, e.g., about 35° C. to about 38° C., about 36° C. to about 39° C., about 36.5° C. to about 39° C., about 36.5° C. to about 37.5° C., or about 36.5° C. to about 38° C.). In some embodiments, the culturing and/or cultivating is carried out at a temperature of about 37° C.

In some embodiments, the culturing and/or cultivating of the mammary cells and/or plasma cells for the cell construct is carried out at an atmospheric concentration of CO₂ of about 4% to about 6%, e.g., an atmospheric concentration of CO₂ of about 4%, 4.25%, 4.5%, 4.75%, 5%, 5.25%, 5.5%, 5.75%, or 6% or any value or range therein, e.g., about 4% to about 5.5%, about 4.5% to about 6%, about 4.5% to about 5.5%, or about 5% to about 6%). In some embodiments, the culturing and/or cultivating is carried out at an atmospheric concentration of CO₂ of about 5%.

In some embodiments, the culturing and/or cultivating of the mammary cells and/or the plasma cells for the cell construct comprises culturing and/or cultivating in a culture medium that is exchanged about every day to about every 10 days (e.g., every 1 day, every 2 days, every 3 days, every 4 days, every 5 days, every 6 days, every 7 days, every 8 days, every 9 days, every 10 days, or any value or range therein, e.g., about every day to every 3 days, about every 3 days to every 10 days, about every 2 days to every 5 days). In some embodiments, the culturing and/or cultivating further comprises culturing in a culture medium that is exchanged about every day to about every few hours to about every 10 days, e.g., about every 1, 2, 3, 4, 5, 6, 7, 8, 9, 10, 11, 12, 13, 14, 15, 16, 17, 18, 19, 20, 21, 22, 23, or 24 hours to about every 1, 2, 3, 4, 5, 6, 7, 8, 9, or 10 days or any value or range therein. For example, in some embodiments, the culturing and/or cultivating further comprises culturing and/or cultivating in a culture medium that is exchanged about every 12 hours to about every 10 days, about every 10 hours to about every 5 days, or about every 5 hours to about every 3 days.

In some embodiments, the cell construct is stored in a freezer or in liquid nitrogen. The storage temperature depends on the desired storage length. For example, freezer temperature (e.g., storage at a temperature of about 0° C. to about −80° C. or less, e.g., about 0° C., −10° C., −20° C., −30° C., −40° C., −50° C., −60° C., −70° C., −80° C., −90° C., −100° C. or any value or range therein) may be used if the cells are to be used within 6 months (e.g., within 1, 2, 3, 4, 5, or 6 months). For example, liquid nitrogen may be used (e.g., storage at a temperature of −100° C. or less (e.g., about −100° C., −110° C., −120° C., −130, −140, −150, −160, −170, −180, −190° C., −200° C., or less) for longer term storage (e.g., storage of 6 months or longer, e.g., 6, 7, 8, 9, 10, 11, or 12 months, or 1, 2, 3, 4, 5, 6 or more years).

In some embodiments, the cell construct comprises a scaffold (as described herein) comprising an upper surface and a lower surface and a continuous monolayer of polarized mammary epithelial cells, a continuous monolayer of a polarized, mixed population of mammary epithelial cells, mammary myoepithelial cells and mammary progenitor cells, and/or a continuous monolayer of polarized immortalized mammary epithelial cells, wherein the continuous monolayer is located on the upper surface of scaffold. In some embodiments, the scaffold comprises a three dimensional scaffold (as described herein) comprising a plurality of fibers that are non-uniformly oriented and/or non-linearly oriented fibers. In some embodiments, the fibers comprise thermoplastic polyurethane and/or polycaprolactone. In some embodiments, the fibers comprise nanofibers.

In some embodiments, the lower surface of the scaffold is adjacent to the basal compartment. In some embodiments, the apical surface of the continuous monolayer is adjacent to the apical compartment. In some embodiments, the continuous monolayer secretes milk and sIgA or IgA through its apical surface into the apical compartment, thereby producing milk comprising IgA and/or sIgA in culture. In some embodiments, the continuous monolayer secretes milk and IgG through its apical surface into the apical compartment, thereby producing milk comprising IgG in culture. In some embodiments, the continuous monolayer secretes milk and sIgM or IgM through its apical surface into the apical compartment, thereby producing milk comprising IgM and/or sIgM in culture.

In some embodiments, the monolayer of mammary cells forms a barrier that divides the apical compartment and the basal compartment, wherein the basal surface of the mammary cells is attached to the scaffold and the apical surface is oriented toward the apical compartment.

In some embodiments, the basal compartment is adjacent to the lower surface of the scaffold. In some embodiments, the basal compartment comprises a culture medium in fluidic contact with the basal surface of the monolayer of mammary epithelial cells (e.g., the polarized monolayer of mammary epithelial cells, the polarized the monolayer of the mixed population of mammary cells, or the polarized monolayer of immortalized mammary epithelial cells).

In some embodiments, the culture medium comprises a carbon source, a chemical buffering system, one or more essential amino acids, one or more vitamins and/or cofactors, and one or more inorganic salts.

In some embodiments, the bioreactor comprises an apical compartment that is adjacent to the apical surface of the monolayer. In some embodiments, the apical compartment is adjacent to the upper surface of the scaffold.

In some embodiments, the bioreactor maintains a temperature of about 27° C. to about 39° C. (e.g., a temperature of about 27° C., 28° C., 29° C., 30° C., 31° C., 32° C., 33° C., 34° C., 35° C., 35° C., 35.5° C., 36° C., 36.5° C., 37° C., 37.5° C., 38° C., 38.5° C. or about 39° C., or any value or range therein, e.g., about 27° C. to about 38° C., about 36° C. to about 39° C., about 36.5° C. to about 39° C., about 36.5° C. to about 37.5° C., or about 36.5° C. to about 38° C.). In some embodiments, the bioreactor maintains a temperature of about 37° C.

In some embodiments, the bioreactor has an atmospheric concentration of CO₂ of about 4% to about 6%, e.g., an atmospheric concentration of CO₂ of about 4%, 4.25%, 4.5%, 4.75%, 5%, 5.25%, 5.5%, 5.75%, or 6% or any value or range therein, e.g., about 4% to about 5.5%, about 4.5% to about 6%, about 4.5% to about 5.5%, or about 5% to about 6%). In some embodiments, the bioreactor has an atmospheric concentration of CO₂ of about 5%.

In some embodiments, the bioreactor has an atmospheric concentration of CO₂ of about 4% to about 6%, e.g., an atmospheric concentration of CO₂ of about 4%, 4.25%, 4.5%, 4.75%, 5%, 5.25%, 5.5%, 5.75%, or 6% or any value or range therein, e.g., about 4% to about 5.5%, about 4.5% to about 6%, about 4.5% to about 5.5%, or about 5% to about 6%). In some embodiments, the bioreactor has an atmospheric concentration of CO₂ of about 5%.

In some embodiments, the method comprises monitoring the concentration of dissolved O₂ and CO₂. In some embodiments, the concentration of dissolved O₂ is maintained between about 10% to about 25% or any value or range therein (e.g., about 10, 11, 12, 13, 14, 15, 16, 17, 18, 19, 20, 21, 22, 23, 24, or 25%). For example, in some embodiments, the concentration of dissolved O₂ is maintained between about 12% to about 25%, about 15% to about 22%, about 10% to about 20%, about 15%, about 20%, or about 22%. In some embodiments, the concentration of CO₂ is maintained between about 4% to about 6%, e.g., a concentration of CO₂ of about 4%, 4.25%, 4.5%, 4.75%, 5%, 5.25%, 5.5%, 5.75%, or 6% or any value or range therein, e.g., about 4% to about 5.5%, about 4.5% to about 6%, about 4.5% to about 5.5%, or about 5% to about 6%). In some embodiments, the concentration of CO₂ is maintained at about 5%.

In some embodiments, the culture medium is exchanged about every day to about every 10 days (e.g., every 1 day, every 2 days, every 3 days, every 4 days, every 5 days, every 6 days, every 7 days, every 8 days, every 9 days, every 10 days, or any value or range therein, e.g., about every day to every 3 days, about every 3 days to every 10 days, about every 2 days to every 5 days). In some embodiments, the culture medium is exchanged about every day to about every few hours to about every 10 days, e.g., about every 1, 2, 3, 4, 5, 6, 7, 8, 9, 10, 11, 12, 13, 14, 15, 16, 17, 18, 19, 20, 21, 22, 23, or 24 hours to about every 1, 2, 3, 4, 5, 6, 7, 8, 9, or 10 days or any value or range therein. For example, in some embodiments, the culture medium is exchanged about every 12 hours to about every 10 days, about every 10 hours to about every 5 days, or about every 5 hours to about every 3 days.

In some embodiments, the method comprises monitoring the glucose concentration and/or rate of glucose consumption in the culture medium and/or in the lactogenic culture medium. In some embodiments, the prolactin is added when the rate of glucose consumption in the culture medium is steady state.

In some embodiments, the method further comprises applying transepithelial electrical resistance (TEER) to measure the maintenance of the monolayer of epithelial cells. TEER measures a voltage difference between the fluids (e.g., media) in two compartments (e.g., between the apical and basal compartments), wherein if the barrier between the compartments loses integrity, the fluids in the two compartments may mix. When there is fluid mixing, the voltage difference will be reduced or eliminated; a voltage difference indicates that the barrier is intact. In some embodiments, upon detection of a loss of voltage by TEER, a scaffold (e.g., a Transwell® filter, a microstructured bioreactor, a decellularized tissue, a hollow fiber bioreactor, etc.) is reinoculated with additional cells and allowed time to reestablish a barrier (e.g., a monolayer) before resuming production of the cultured milk product (e.g., milk production). In some embodiments, the TEER (as measured in Ohms*cm²) is from about −80 Ohms*cm² to about 200 Ohms*cm². In some embodiments, the TEER is at least about 0 Ohms*cm². In some embodiments, the TEER is at least about 10 Ohms*cm². In some embodiments, the TEER is at least about 20 Ohms*cm². In some embodiments, the TEER is at least about 30 Ohms*cm². In some embodiments, the TEER is at least about 40 Ohms*cm². In some embodiments, the TEER is at least about 50 Ohms*cm². In some embodiments, the TEER is at least about 60 Ohms*cm². In some embodiments, the TEER is at least about 70 Ohms*cm². In some embodiments, the TEER is at least about 80 Ohms*cm². In some embodiments, the TEER is at least about 90 Ohms*cm². In some embodiments, the TEER is at least about 100 Ohms*cm². In some embodiments, the TEER is at least about 150 Ohms*cm². In some embodiments, the TEER is at least about 200 Ohms*cm². In some embodiments, the TEER increases with the duration of cell culture. In some embodiments, a scaffold with extra cellular matrix (ECM)-coated TPU has a higher average TEER value than a scaffold with ECM-coated PCL, ECM-coated PET, uncoated TPU, uncoated PCL, or uncoated PET.

In some embodiments, the method further comprises collecting the cultured milk product from the apical compartment to produce collected cultured milk product. In some embodiments, the collecting is via a port, via gravity, and/or via a vacuum. In some embodiments, a vacuum is attached to a port.

### Basal Culture Media and Lactogenic Media

In some embodiments, the culture medium comprises a carbon source, a chemical buffering system, one or more essential amino acids, one or more vitamins and/or cofactors, and one or more inorganic salts. In some embodiments, the carbon source, chemical buffering system, one or more essential amino acids, one or more vitamins and/or cofactors, and/or one or more inorganic salts are food grade. As used herein, the term “culture medium”, “culture media”, “cell medium”, and/or “cell media” may be used interchangeably.

In some embodiments, the culture medium is lactogenic culture medium. In some embodiments, the culture medium further comprises prolactin (e.g., mammalian prolactin, e.g., human prolactin), linoleic and alpha-linoleic acid, estrogen and/or progesterone. For example, in some embodiments, the culture medium comprises prolactin (or prolactin is added) in an amount from about 20 ng/mL to about 200 ng/L of culture medium, e.g., about 20, 30, 40, 50, 60, 70, 80, 90, 100, 110, 120, 130, 140, 150, 160, 170, 180, 190, or 200 ng/mL or any value or range therein. In some embodiments, the culture medium comprises prolactin (or prolactin is added) in an amount from about 20 ng/mL to about 195 ng/mL, about 50 ng/ml to about 150 ng/mL, about 25 ng/ml to about 175 ng/mL, about 45 ng/mL to about 200 ng/mL, or about 75 ng/mL to about 190 ng/ml of culture medium. In some embodiments, the culture medium further comprises other factors to improve efficiency, including, but not limited to, insulin, an epidermal growth factor, and/or a hydrocortisone.

In some embodiments, the culture medium comprises a carbon source in an amount from about 1 g/L to about 15 g/L of culture medium (e.g., about 1, 2, 3, 4, 5, 6, 7, 8, 9, 10, 11, 12, 13, 14 or 15 g/L or any value or range therein), or about 1, 2, 3, 4, 5 or 6 g/L to about 7, 8, 9, or 10, 11, 12, 13, 14 or 15 g/L of the culture medium. Non-limiting examples of a carbon source include glucose and/or pyruvate. For example, in some embodiments, the culture medium comprises glucose in an amount from about Ig/L to about 12 g/L of culture medium, e.g., about 1, 2, 3, 4, 5, 6, 7, 8, 9, 10, 11, or 12 g/L or any value or range therein. In some embodiments, the culture medium comprises glucose in an amount from about 1 g/L to about 6 g/L, about 4 g/L to about 12 g/L, about 2.5 g/L to about 10.5 g/L, about 1.5 g/L to about 11.5 g/L, or about 2 g/L to about 10 g/L of culture medium. In some embodiments, the culture medium comprises glucose in an amount from about 1, 2, 3, or 4 g/L to about 5, 6, 7, 8, 9, 10, 11, or 12 g/L or about 1, 2, 3, 4, 5, or 6 g/L to about 7, 8, 9, 10, 11, or 12 g/L. In some embodiments, the culture medium comprises pyruvate in an amount from about 5 g/L to about 15 g/L of culture medium, e.g., about 5, 6, 7, 8, 9, 10, 11, 12, 13, 14, or 15 g/L or any value or range therein. In some embodiments, the culture medium comprises pyruvate in an amount from about 5 g/L to about 14.5 g/L, about 10g/L to about 15 g/L, about 7.5 g/L to about 10.5 g/L, about 5.5 g/L to about 14.5 g/L, or about 8 g/L to about 10 g/L of culture medium. In some embodiments, the culture medium comprises pyruvate in an amount from about 5, 6, 7, or 8 g/L to about 9, 10, 11, 12, 13, 14 or 15 g/L or about 5, 6, 7, 8, 9, or 10 g/L to about 11, 12, 13, 14 or 15 g/L.

In some embodiments, the culture medium comprises a chemical buffering system in an amount from about 1 g/L to about 4 g/L (e.g., about 1, 1.5, 2, 2.5, 3, 3.5, or 4 g/L or any value or range therein) of culture medium or about 10 mM to about 25 mM (e.g., about 10, 11, 12, 13, 14, 15, 16, 17, 18, 19, 20, 21, 22, 23, 24, or 25 mM or any value or range therein). In some embodiments, the chemical buffering system includes, but is not limited to, sodium bicarbonate and/or 4-(2-hydroxyethyl)-l-piperazineethanesulfonic acid (HEPES). For example, in some embodiments, the culture medium comprises sodium bicarbonate in an amount from about 1 g/L to about 4 g/L of culture medium, e.g., about 1, 1.5, 2, 2.5, 3, 3.5, or 4 g/L or any value or range therein. In some embodiments, the culture medium comprises sodium bicarbonate in an amount from about 1 g/L to about 3.75 g/L, about 1.25 g/L to about 4 g/L, about 2.5 g/L to about 3 g/L, about 1.5 g/L to about 4 g/L, or about 2 g/L to about 3.5 g/L of culture medium. In some embodiments, the culture medium comprises HEPES in an amount from about 10 mM to about 25 mM, e.g., about 10, 11, 12, 13, 14, 15, 16, 17, 18, 19, 20, 21, 22, 23, 24, or 25 mM or any value or range therein. In some embodiments, the culture medium comprises HEPES in an amount from about 11 mM to about 25 mM, about 10 mM to about 20 mM, about 12.5 mM to about 22.5 mM, about 15 mM to about 20.75 mM, or about 10 mM to about 20 mM.

In some embodiments, the culture medium comprises one or more essential amino acids in an amount from about 0.5 mM to about 5 mM (e.g., about 0.5, 1, 1.5, 2, 2.5, 3, 3.5, 4, 4.5, or 5 mM or any value or range therein) or about 0.5, 1, 1.5, 2 mM to about 2.5, 3, 3.5, 4, 4.5, or 5 mM. In some embodiments, the one or more essential amino acids is histidine, isoleucine, leucine, lysine, methionine, phenylalanine, threonine, tryptophan, valine, and/or arginine. For example, in some embodiments, the culture medium comprises arginine in an amount from about 0.5 mM to about 5 mM, e.g., about 0.5, 1, 1.5, 2, 2.5, 3, 3.5, 4, 4.5, or 5 mM or any value or range therein. In some embodiments, the culture medium comprises an essential amino acids in an amount from about 0.5 mM to about 4.75 mM, about 2 mM to about 3.5 mM, about 0.5 mM to about 3.5 mM, about 1 mM to about 5 mM, or about 3.5 mM to about 5 mM.

In some embodiments, the culture medium comprises one or more vitamins and/or cofactors in an amount from about 0.01 μM to about 50 μM (e.g., about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 3, 4, 5, 6, 7,8,9, 10, 12.5, 15, 17.5,20, 25, 30, 35, 40, 45, 46, 47, 48, 49, 49.025, 49.05, 49.075, or 50 UM or any value or range therein) or about 0.01, 0.02, 0.03, 0.04, 0.05, 0.06, 0.07, 0.08, 0.09, 0.1, 0.2, 0.3, 0.4, 0.5, 0.6, 0.7, 0.8, or 0.9 μM to about 1, 1.1, 1.2, 1.3, 1.4, 1.5, 1.6, 1.7, 1.8, 1.9, 2, 2.1, 2.2, 2.3, 2.4, 2.5, 3, 4, 5, 6 UM or about 0.02, 0.025, 0.05, 0.075, 1, 1.5, 2, 3, 4, 5, 6, 7, 8, 9, 10 μM to about 12.5, 15, 17.5, 20, 25, 30, 35, 40, 45, 46, 47, 48, 49, 49.025, 49.05, 49.075, or 50 μM. In some embodiments, one or more vitamins and/or cofactors include, but are not limited to, thiamine and/or riboflavin. For example, in some embodiments, the culture medium comprises thiamine in an amount from about 0.025 μM to about 50 μM, e.g., about 0.025, 0.05, 0.075, 1, 1.5, 2, 3, 4, 5, 6, 7, 8, 9, 10, 12.5, 15, 17.5, 20, 25, 30, 35, 40, 45, 46, 47, 48, 49, 49.025, 49.05, 49.075, or 50 μM or any value or range therein. In some embodiments, the culture medium comprises thiamine in an amount from about 0.025 μM to about 45.075 μM, about 1 μM to about 40 μM, about 5 μM to about 35.075 μM, about 10 μM to about 50 μM, or about 0.05 μM to about 45.5 μM. In some embodiments, the culture medium comprises riboflavin in an amount from about 0.01 μM to about 3 μM, e.g., about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or 3 μM or any value or range therein. In some embodiments, the culture medium comprises riboflavin in an amount from about 0.01 μM to about 2.05 μM, about 1 μM to about 2.95 μM, about 0.05 μM to about 3 μM, about 0.08 UM to about 1.55 μM, or about 0.05 μM to about 2.9 μM.

In some embodiments, the culture medium comprises one or more inorganic salts in an amount from about 100 mg/L to about 150 mg/L of culture medium (e.g., about 100, 105, 110, 115, 120, 125, 130, 135, 140, 145, or 150 mg/L or any value or range therein) or about 100 mg/L to about 150 mg/L of culture medium (e.g., about 100, 105, 110, 115, 120, 125, 130, 135, 140, 145, or 150 mg/L or any value or range therein). In some embodiments, one or more inorganic salts include, but are not limited to, calcium and/or magnesium. For example, in some embodiments, the culture medium comprises calcium in an amount from about 100 mg/L to about 150 mg/L of culture medium, e.g., about 100, 105, 110, 115, 120, 125, 130, 135, 140, 145, or 150 mg/L or any value or range therein. In some embodiments, the culture medium comprises arginine in an amount from about 100 mg/L to about 125 mg/L, about 105 mg/L to about 150 mg/L, about 120 mg/L to about 130 mg/L, or about 100 mg/L to about 145 mg/L of culture medium. In some embodiments, the culture medium comprises magnesium in an amount from about 0.01 mM to about 1 mM, e.g., about 0.01,0.02, 0.03, 0.04, 0.05, 0.06, 0.07, 0.08, 0.09, 0.1, 0.2, 0.3, 0.4, 0.5, 0.6, 0.7, 0.8, 0.9, 0.91, 0.92, 0.93, 0.94, 0.95, 0.96, 0.97, 0.98, 0.99, or 1 mM or any value or range therein. In some embodiments, the culture medium comprises magnesium in an amount from about 0.05 mM to about 1 mM, about 0.01 mM to about 0.78 mM, about 0.5 mM to about 1 mM, about 0.03 mM to about 0.75 mM, or about 0.25 mM to about 0.95 mM.

In some embodiments, the culture medium comprises a carbon source in an amount from about 1 g/L to about 15 g/L of culture medium (e.g., about 1, 2, 3, 4, 5, 6, 7, 8, 9, 10, 11, 12, 13, 14 or 15 g/L or any value or range therein), or about 1, 2, 3, 4, 5 or 6 g/L to about 7, 8, 9, or 10, 11, 12, 13, 14 or 15 g/L of the culture medium. In some embodiments, the carbon source includes, but is not limited to, glucose and/or pyruvate. For example, in some embodiments, the culture medium comprises glucose in an amount from about 1 g/L to about 12 g/L of culture medium, e.g., about 1, 2, 3, 4, 5, 6, 7, 8, 9, 10, 11, or 12 g/L or any value or range therein. In some embodiments, the culture medium comprises glucose in an amount from about 1 g/L to about 6 g/L, about 4 g/L to about 12 g/L, about 2.5 g/L to about 10.5 g/L, about 1.5 g/L to about 11.5 g/L, or about 2 g/L to about 10 g/L of culture medium. In some embodiments, the culture medium comprises pyruvate at an amount of about 5 g/L to about 15 g/L of culture medium, e.g., about 5, 6, 7, 8, 9, 10, 11, 12, 13, 14, or 15 g/L or any value or range therein. In some embodiments, the culture medium comprises pyruvate in an amount from about 5 g/L to about 14.5 g/L, about 10 g/L to about 15 g/L, about 7.5 g/L to about 10.5 g/L, about 5.5 g/L to about 14.5 g/L, or about 8 g/L to about 10 g/L of culture medium.

In some embodiments, the culture medium comprises a chemical buffering system in an amount from about 1 g/L to about 4 g/L (e.g., about 1, 1.5, 2, 2.5, 3, 3.5, or 4 g/L or any value or range therein) of culture medium or about 10 mM to about 25 mM (e.g., about 10, 11, 12, 13, 14, 15, 16, 17, 18, 19, 20, 21, 22, 23, 24, or 25 mM or any value or range therein). In some embodiments, the chemical buffering system includes, but is not limited to, sodium bicarbonate and/or HEPES. For example, in some embodiments, the culture medium comprises sodium bicarbonate in an amount from about 1 g/L to about 4 g/L of culture medium, e.g., about 1, 1.5, 2, 2.5, 3, 3.5, or 4 g/L or any value or range therein. In some embodiments, the culture medium comprises sodium bicarbonate in an amount from about 1 g/L to about 3.75 g/L, about 1.25 g/L to about 4 g/L, about 2.5 g/L to about 3 g/L, about 1.5 g/L to about 4 g/L, or about 2 g/L to about 3.5 g/L of culture medium. In some embodiments, the culture medium comprises HEPES in an amount from about 10 mM to about 25 mM, e.g., about 10, 11, 12, 13, 14, 15, 16, 17, 18, 19, 20, 21, 22, 23, 24, or 25 mM or any value or range therein. In some embodiments, the culture medium comprises HEPES in an amount from about 1 mM to about 25 mM, about 10 mM to about 20 mM, about 12.5 mM to about 22.5 mM, about 15 mM to about 20.75 mM, or about 10 mM to about 20 mM.

In some embodiments, the culture medium comprises one or more essential amino acids in an amount from about 0.5 mM to about 5 mM (e.g., about 0.5, 1, 1.5, 2, 2.5, 3, 3.5, 4, 4.5, or 5 mM or any value or range therein) or about 0.5, 1, 1.5, 2 mM to about 2.5, 3, 3.5, 4, 4.5, or 5 mM. In some embodiments, one or more essential amino acids is arginine and/or cysteine. For example, in some embodiments, the culture medium comprises arginine in an amount from about 0.5 mM to about 5 mM, e.g., about 0.5, 1, 1.5, 2, 2.5, 3, 3.5, 4, 4.5, or 5 mM or any value or range therein. In some embodiments, the culture medium comprises arginine in an amount from about 0.5 mM to about 4.75 mM, about 2 mM to about 3.5 mM, about 0.5 mM to about 3.5 mM, about 1 mM to about 5 mM, or about 3.5 mM to about 5 mM. For example, in some embodiments, the culture medium comprises cysteine in an amount from about 0.5 mM to about 5 mM, e.g., about 0.5, 1, 1.5, 2, 2.5, 3, 3.5, 4, 4.5, or 5 mM or any value or range therein. In some embodiments, the culture medium comprises cysteine in an amount from about 0.5 mM to about 4,75 mM, about 2 mM to about 3.5 mM, about 0.5 mM to about 3.5 mM, about 1 mM to about 5 mM, or about 3.5 mM to about 5 mM.

In some embodiments, the culture medium comprises one or more vitamins and/or cofactors in an amount from about 0.01 μM to about 50 μM (e.g., about 0.01, 0.02, 0.03, 0.04, 0.05, 0.06, 0.07, 0.08, 0.09, 0.1,0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 3, 4, 5, 6, 7, 8,9, 10, 12.5, 15, 17.5, 20, 25, 30, 35, 40, 45, 46, 47, 48, 49, 49.025, 49.05, 49.075, or 50 μM or any value or range therein) or about 0.01, 0.02, 0.03, 0.04, 0.05, 0.06, 0.07, 0.08, 0.09, 0.1, 0.2, 0.3, 0.4, 0.5, 0.6, 0.7, 0.8, or 0.9 μM to about 1, 1.1, 1.2, 1.3, 1.4, 1.5, 1.6, 1.7, 1.8, 1.9, 2, 2.1, 2.2, 2.3, 2.4, 2.5, 3, 4, 5, 6 μM or about 0.02, 0.025, 0.05, 0.075, 1, 1.5, 2, 3, 4, 5, 6, 7, 8, 9, 10 μM to about 12.5, 15, 17.5, 20, 25, 30, 35, 40, 45, 46, 47, 48, 49, 49.025, 49.05, 49.075, or 50 μM. In some embodiments, one or more vitamins and/or cofactors includes, but is not limited to, thiamine and/or riboflavin. For example, in some embodiments, the culture medium comprises thiamine in an amount from about 0.025 μM to about 50 μM, e.g., 0.025, 0.05, 0.075, 1, 1.5, 2, 3, 4, 5, 6, 7, 8, 9, 10, 12.5, 15, 17.5, 20, 25, 30, 35, 40, 45, 46, 47, 48, 49, 49.025, 49.05, 49.075, or 50 μM or any value or range therein. In some embodiments, the culture medium comprises thiamine in an amount from about 0.025μM to about 45.075 μM, about 1 μM to about 40 μM, about 5 μM to about 35.075 μM, about 10 μM to about 50 μM, or about 0.05 μM to about 45.5 μM. In some embodiments, the culture medium comprises riboflavin in an amount from about 0.01 μM to about 3 μM, e.g., 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or 3 μM or any value or range therein. In some embodiments, the culture medium comprises riboflavin in an amount from about 0.01 μM to about 2.05 M, about 1 μM to about 2.95 μM, about 0.05 μM to about 3 μM, about 0.08 UM to about 1.55 μM, or about 0.05 μM to about 2.9 pM.

In some embodiments, the culture medium comprises one or more inorganic salts in an amount from about 100 mg/L to about 150 mg/L of culture medium (e.g., about 100, 105, 110, 115, 120, 125, 130, 135, 140, 145, or 150 mg/L or any value or range therein) or about 100 mg/L to about 150 mg/L of culture medium (e.g., about 100, 105, 110, 115, 120, 125, 130, 135, 140, 145, or 150 mg/L or any value or range therein). In some embodiments, exemplary one or more inorganic salts is calcium and/or magnesium. For example, in some embodiments, the culture medium comprises calcium in an amount from about 100 mg/L to about 150 mg/L of culture medium, e.g., about 100, 105, 110, 115, 120, 125, 130, 135, 140, 145, or 150 mg/L or any value or range therein. In some embodiments, the culture medium comprises arginine in an amount from about 100 mg/L to about 125 mg/L, about 105 mg/L to about 150 mg/L, about 120 mg/L to about 130 mg/L, or about 100 mg/L to about 145 mg/L of culture medium. In some embodiments, the culture medium comprises magnesium in an amount from about 0.01 mM to about 1 mM, e.g., about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 0.91, 0.92, 0.93, 0.94, 0.95, 0.96, 0.97, 0.98, 0.99, or 1 mM or any value or range therein. In some embodiments, the culture medium comprises magnesium in an amount from about 0.05 mM to about 1 mM, about 0.01 mM to about 0.78 mM, about 0.5 mM to about 1 mM, about 0.03 mM to about 0.75 mM, or about 0.25 mM to about 0.95 mM.

In some embodiments, the carbon source, chemical buffering system, one or more essential amino acids, one or more vitamins and/or cofactors, and/or one or more inorganic salts is food grade.

In some embodiments, the culture medium is lactogenic culture medium, e.g., the culture medium further comprises prolactin (e.g., mammalian prolactin, e.g., human prolactin). For example, in some embodiments, the culture medium comprises prolactin (or prolactin is added) in an amount from about 20 ng/mL to about 200 ng/L of culture medium, e.g., about 20, 30, 40, 50, 60, 70, 80, 90, 100, 110, 120, 130, 140, 150, 160, 170, 180, 190, or 200 ng/ml or any value or range therein. In some embodiments, the culture medium comprises prolactin (or prolactin is added) in an amount from about 20 ng/ml to about 195 ng/ml, about 50 ng/ml to about 150 ng/ml, about 25 ng/ml to about 175 ng/ml, about 45 ng/ml to about 200 ng/ml, or about 75 ng/ml to about 190 ng/ml of culture medium. In some embodiments, the methods further comprise adding prolactin to the culture medium, thereby providing a lactogenic culture medium.

In some embodiments, the prolactin is produced by a microbial cell and/or a human cell expressing a recombinant prolactin (e.g., a prolactin comprising a substitution of a serine residue at position 179 of the prolactin gene with aspartate (S179D), e.g., S179D-prolactin). In some embodiments, adding prolactin to the culture medium comprises conditioning culture medium by culturing cells that express and secrete prolactin, and applying the conditioned culture medium comprising prolactin to the basal surface of the monolayer of mammary cells (e.g., mammary epithelial cells, mammary myoepithelial cells and mammary progenitor cells).

In some embodiments, the culture medium further comprises other factors to improve efficiency, including, but not limited to, insulin, an epidermal growth factor, and/or a hydrocortisone. In some embodiments, the methods of the present invention further comprise adding other factors (e.g., insulin, an epidermal growth factor, and/or a hydrocortisone) to the culture medium, e.g., to improve efficiency.

Having described the present disclosure, the same will be explained in greater detail in the following examples, which are included herein for illustration purposes only, and which are not intended to be limiting to the disclosure.

### EXAMPLES

The Examples that follow are illustrative of specific embodiments of the disclosure, and various uses thereof. They are set forth for explanatory purposes only and should not be construed as limiting the scope of the disclosure in any way.

### Example 1

A cell culture system designed for the collection of milk should support compartmentalized secretion of the product such that the milk is not exposed to the media that provides nutrients to the cells. In the body, milk-producing epithelial cells line the interior surface of the mammary gland as a continuous monolayer. The monolayer is oriented such that the basal surface is attached to an underlying basement membrane, while milk is secreted from the apical surface and stored in the luminal compartment of the gland, or alveolus, until it is removed during milking or feeding. Tight junctions along the lateral surfaces of the cells ensure a barrier between the underlying tissues and the milk located in the alveolar compartment. Therefore, in vivo, the tissue of the mammary gland is arranged such that milk secretion is compartmentalized, with the mammary epithelial cells themselves establishing the interface and maintaining the directional absorption of nutrients and secretion of milk.

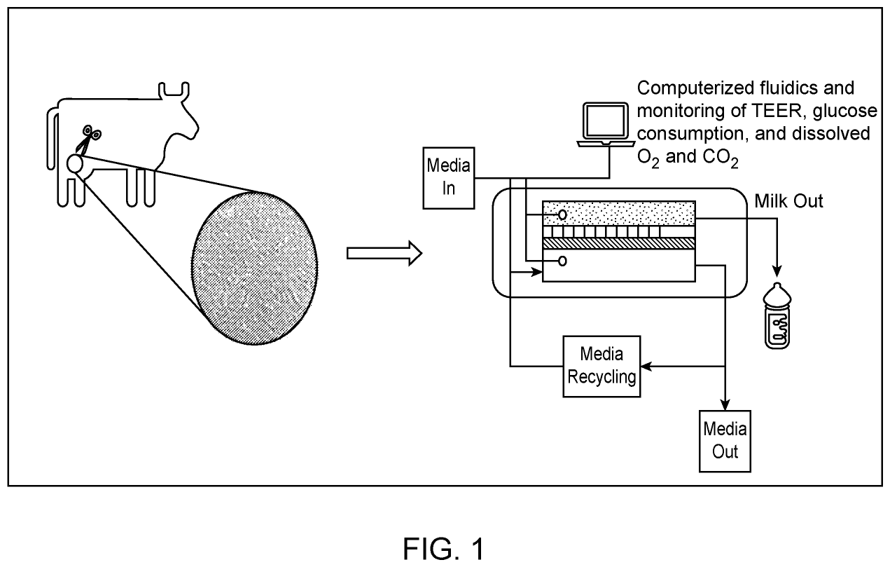

The present disclosure describes a cell culture apparatus that recapitulates the compartmentalizing capability of the mammary gland that is used to collect milk from mammary epithelial cells grown outside of the body. Such an apparatus can include a scaffold to support the proliferation of mammary cells at the interface between two compartments, such that the epithelial monolayer provides a physical boundary between the nutrient medium and the secreted milk. In addition to providing a surface for growth, the scaffold provides spatial cues that guide the polarization of the cells and ensures the directionality of absorption and secretion. This invention describes the preparation, cultivation, and stimulation of mammary epithelial cells in a compartmentalizing cell culture apparatus for the production and collection of milk for nutritional use (see e.g., FIG. 1).

Preparation of mammary epithelial cells. Mammary epithelial cells are obtained from surgical explants of dissected mammary tissue (e.g., breast, udder, teat), biopsy sample, or raw breastmilk. Generally, after surgical dissection of the mammary tissue, any fatty or stromal tissue is manually removed under aseptic conditions, and the remaining tissue of the mammary gland is enzymatically digested with collagenase and/or hyaluronidase prepared in a chemically defined nutrient media, which should be composed of ingredients that are “generally recognized as safe” (GRAS). The sample is maintained at 37° C. with gentle agitation. After digestion, a suspension of single cells or organoids is collected, either by centrifugation or by pouring the sample through a sterile nylon cell strainer. The cell suspension is then transferred to a tissue culture plate coated with appropriate extracellular matrix components (e.g., collagen, laminin, fibronectin).

Alternatively, explant specimens can be processed into small pieces, for example by mincing with a sterile scalpel. The tissue pieces are plated onto a suitable surface such as a gelatin sponge or a plastic tissue culture plate coated with appropriate extracellular matrix.

The plated cells are maintained at 37° C. in a humidified incubator with an atmosphere of 5% CO₂. During incubation, the media is exchanged about every 1 to 3 days and the cells are sub-cultured until a sufficient viable cell number is achieved for subsequent processing, which includes preparation for storage in liquid nitrogen; development of immortalized cell lines through the stable transfection of genes such as SV40, TERT, or other genes associated with senescence; isolation of mammary epithelial, myoepithelial, and stem/progenitor cell types by, for example, fluorescence-activated cell sorting; and/or introduction into a compartmentalizing tissue culture apparatus for the production and collection of milk for human consumption.

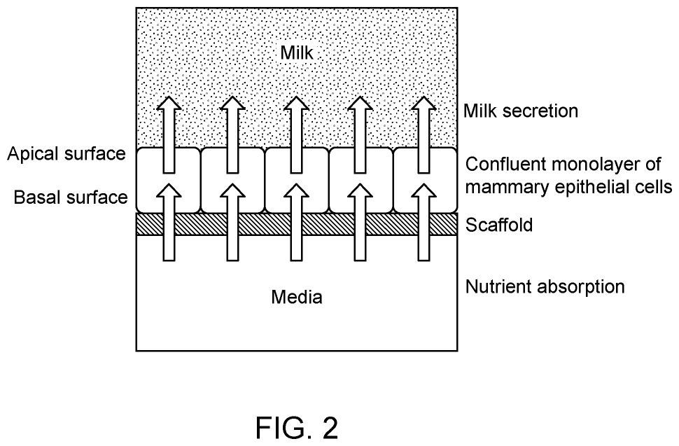

Cultivation of mammary epithelial cells for the production of milk. Milk for nutritional use is produced by mammary epithelial cells isolated as described above and cultured in a format that supports compartmentalized secretion such that separation between the nutrient medium and the product is maintained. The system relies on the ability of mammary epithelial cells to establish a continuous monolayer with appropriate apical-basal polarity when seeded onto an appropriate scaffold positioned at the interface between the apical compartment, into which milk is secreted, and the basal compartment, through which nutrient media is provided (see, e.g., FIG. 2). Transwell® filters placed in tissue culture plates, as well as bioreactors based on hollow fiber or microstructured scaffolds, for example, are used to support these characteristics.

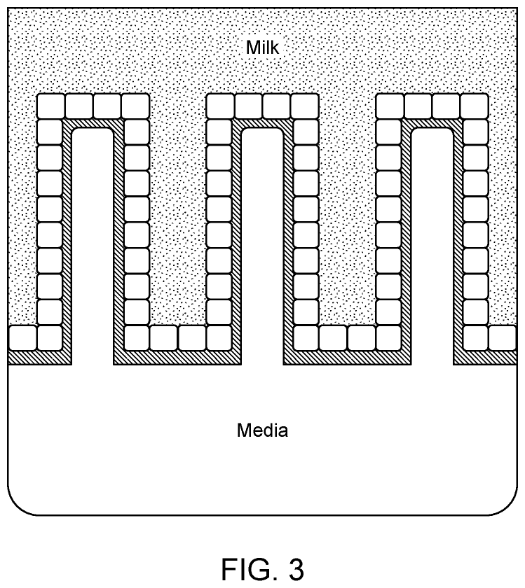

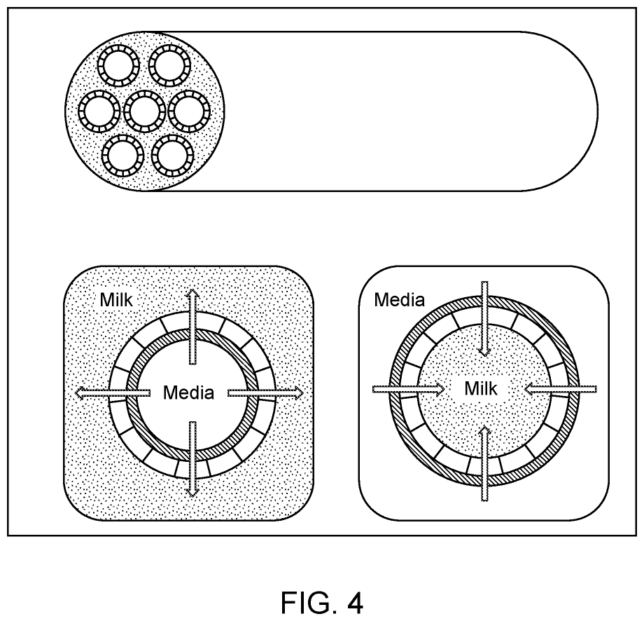

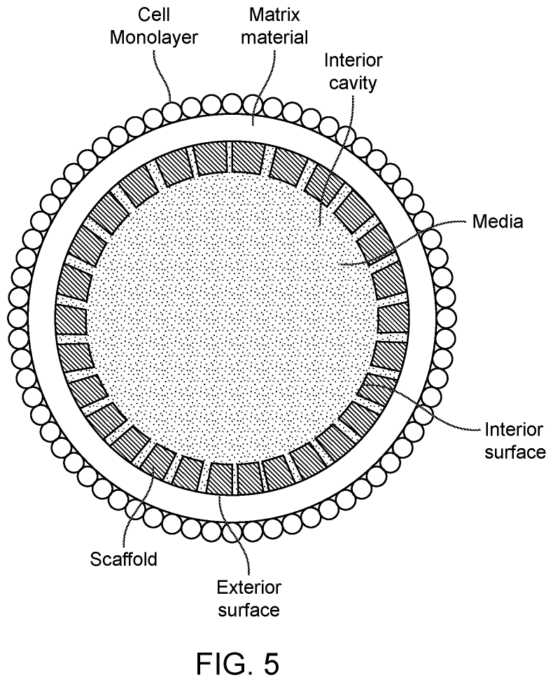

Following the isolation and expansion of mammary epithelial cells, the cells are suspended in a chemically defined nutrient medium composed of food-grade components and inoculated into a culture apparatus that has been pre-coated with a mixture of extracellular matrix proteins, such as collagen, laminin, and/or fibronectin. The cell culture apparatus is any design that allows for the compartmentalized absorption of nutrients and secretion of product from a polarized, confluent, epithelial monolayer. Examples include hollow fiber and microstructured scaffold bioreactors (see, e.g., FIGS. 3 and 4, respectively). Alternatives include other methods of 3-dimensional tissue culture, such as the preparation of decellularized mammary gland as a scaffold, repopulated with stem cells to produce a functional organ in vitro, or collection of milk from the lumen of mammary epithelial cell organoids or “mammospheres” grown either in a hydrogel matrix or in suspension.

The apparatus includes sealed housing that maintains a temperature of about 37° C. in a humidified atmosphere of about 5% CO₂. Glucose uptake is monitored to evaluate the growth of the culture as the cells proliferate within the bioreactor. Stabilization of glucose consumption indicates that the cells have reached a confluent, contact-inhibited state. The integrity of the monolayer is ensured using transepithelial electrical resistance. Sensors monitor concentrations of dissolved 02 and CO₂ in the media at multiple locations. A computerized pump circulates media through the bioreactor at a rate that balances the delivery of nutrients with the removal of metabolic waste such as ammonia and lactate. Media can be recycled through the system after removal of waste using Lactate Supplementation and Adaptation technology (Freund et al. 2018Int J Mol Sci. 19(2)) or by passing through a chamber of packed zeolite.

Stimulation of milk production. In vivo and in cultured mammary epithelial cells, the production and secretion of milk is stimulated by prolactin. In culture, prolactin can be supplied exogenously in the nutrient media at concentrations approximating those observed in the body during lactation, e.g., about 20 ng/mL to about 200 ng/mL. Purified prolactin can be obtained commercially; however, alternative methods of providing prolactin or stimulating lactation are employed, including expression and purification of the recombinant protein from microbial or mammalian cell cultures. Alternatively, conditioned media prepared by culturing cells that express and secrete prolactin can be applied to mammary epithelial cell cultures to stimulate lactation. Bioreactors can be set up in series such that media passing through a culture of cells expressing prolactin or other key media supplements is conditioned prior to exposure to mammary cells grown in a compartmentalizing culture apparatus as described.

Other approaches to upregulate milk production and/or spare the use of exogenous prolactin include molecular manipulation of the signaling pathways that are regulated by binding of prolactin to its receptor on the surface of mammary epithelial cells, such as the following: (a) expression of constructs targeting the posttranslational modification of prolactin; (b) expression of alternative isotypes of the prolactin receptor; (c) expression of a chimeric prolactin receptor in which the extracellular domain is exchanged with the binding site for a different ligand; (d) introduction of a gene encoding a constitutively or conditionally active prolactin receptor or modified versions of its downstream effectors such as STAT5 or Akt; (e) knockout or modification of the PER2 circadian gene; and/or (f) molecular approaches aimed at increasing the rate of nutrient uptake at the basal surface of the mammary epithelial monolayer.

Collection of milk. Secreted milk is collected continuously or at intervals through, for example, a port installed in the apical compartment of the culture apparatus. A vacuum is applied to the port to facilitate collection and also contributes to the stimulation of further production. The collected milk is packaged into sterile containers and sealed for distribution, frozen or lyophilized for storage, or processed for the extraction of specific components.

The present invention provides mammary epithelial cell cultures for the production of milk for nutritional use. In addition to human breast milk, this method may be used to produce milk from other mammalian species, for example, for human consumption or veterinary use. Because it has not been previously possible to produce milk outside the body, this technology may result in novel commercial opportunities, in addition to providing an alternative mode of production for existing products. The social and economic effects of the commercial development of this technology are broad and far reaching. Production of human breast milk from cultured cells may provide a means to address infant malnutrition in food-scarce communities, provide essential nutrients to premature infants who are unable to breastfeed, and offer mothers a new option for feeding their babies that provides optimal nutrition with the convenience of infant formula. Production of cow or goat milk provides an opportunity to reduce the environmental, social, and animal welfare effects of animal agriculture. The process described here addresses an important gap in the emerging field of cellular agriculture and introduces an opportunity to dramatically update the human food supply without compromising our biological and cultural attachment to the most fundamental of our nutrition sources.

### Example 2: Mammary Epithelial Cell Growth Using Fibrous Scaffolds Comprising Thermoplastic Polyurethane (TPU)

Cell growth, specifically primary human mammary epithelial cells (pHMECs), was demonstrated with synthetic fibrous scaffolds comprising a plurality of nanofibers (as described herein) that comprise thermoplastic polyurethane (TPU). The synthetic fibrous scaffolds (comprising the plurality of nanofibers) were made through electrospinning, wherein the electrospun materials were made into sheets that were manually immobilized into a transwell insert (Scaffdex CellCrown™ inserts) for cell culture. Specifically, pHMECs sourced from Lonza and Sigma respectively were cultured for 6 days onto the electrospun fibrous scaffolds (comprising the plurality of nanofibers). Some of the scaffolds were coated with Collagen-IV and Laminin-1, while some of the scaffolds were not coated with Collagen-IV and Laminin-1. The cultured cells were probed for the following biological processes: a) cellular architecture/epithelial monolayer formation through actin immunofluorescence (IF) staining and hematoxylin and eosin (H&E) staining, b) tight junction formation/epithelial differentiation through IF staining for epithelial/tight junction marker E-cadherin staining, c) cell proliferation, and d) epithelial barrier function through fluorescein isothiocyanate-dextran (FITC-dextran) (4 kD) diffusion assay.

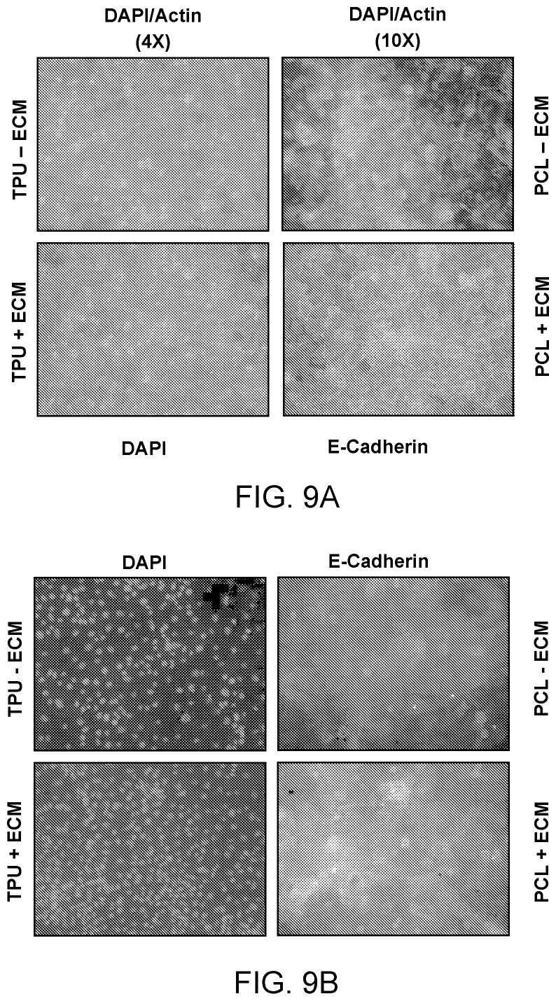

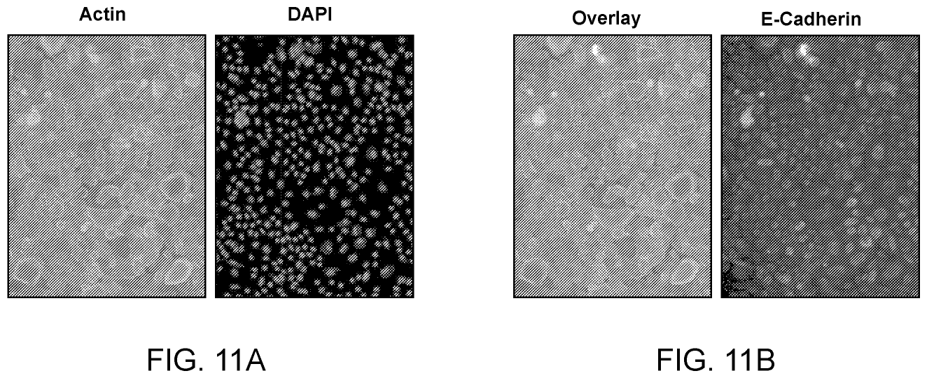

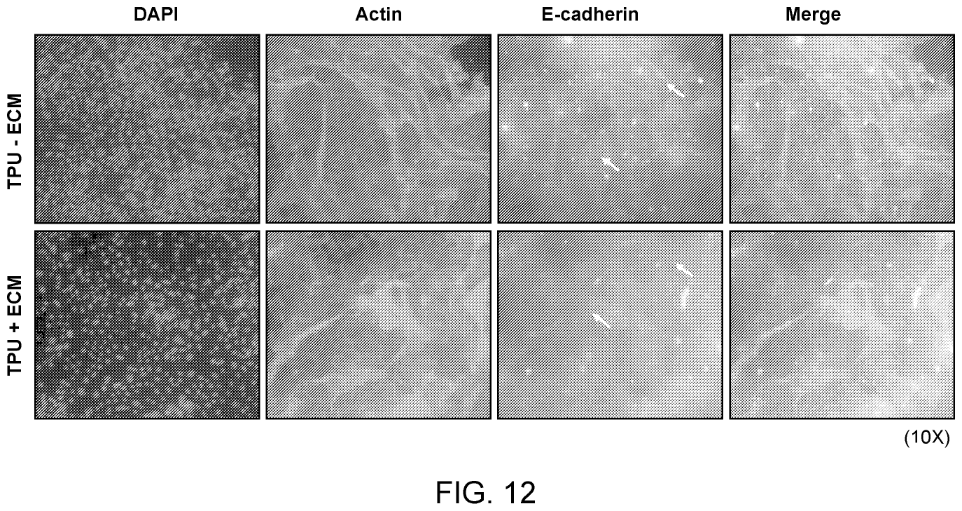

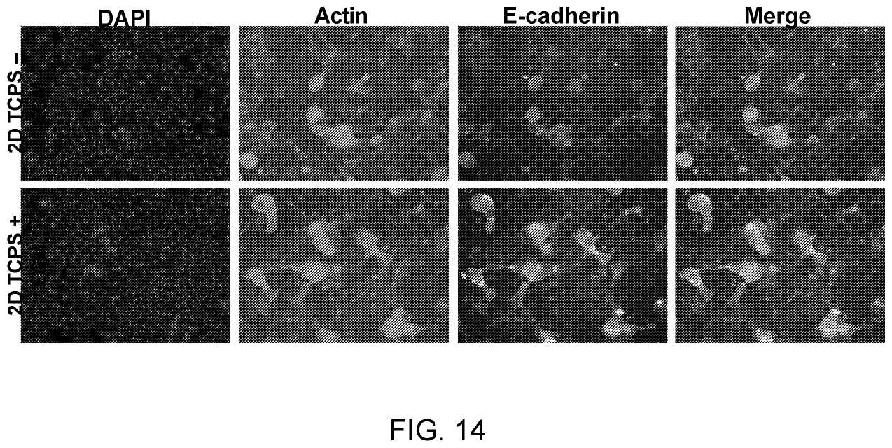

It was observed that Lonza pHMECs form intact cell monolayers on the electrospun fibrous scaffolds (comprising a plurality of nanofibers) made from TPU polymers, both with or without Collagen-IV and Laminin-1 coating, as indicated by actin IF staining (FIG. 9A). The actin staining in Lonza pHMECs also revealed the presence of cortical actin which is indicative of an epithelial phenotype (FIG. 9A). Contrary to Lonza pHMECs cultured on the electrospun fibrous scaffolds, the Lonza pHMECs did not form intact monolayers when cultured on conventional plastic (for example, tissue culture plastic TCP) as revealed by actin staining (at day 3) (FIG. 11A). The cells appeared to be non-uniform in shape and had a more spread morphology with formation of actin stress fibers which indicate deviation away from the epithelial phenotype. Similarly, it was observed that Sigma pHMECs also formed intact cell monolayers on the electrospun fibrous scaffolds made from TPU, both with or without Collagen-IV and Laminin-1, as indicated by actin IF staining (FIG. 12). Cortical actin was also observed in Sigma pHMECs cultured on TPU the electrospun scaffolds, which is indicative of an epithelial phenotype (FIG. 12). Unlike Lonza pHMECs, Sigma pHMECs also form monolayer-like structures exhibiting cortical actin when cultured on conventional plastic (FIG. 14).

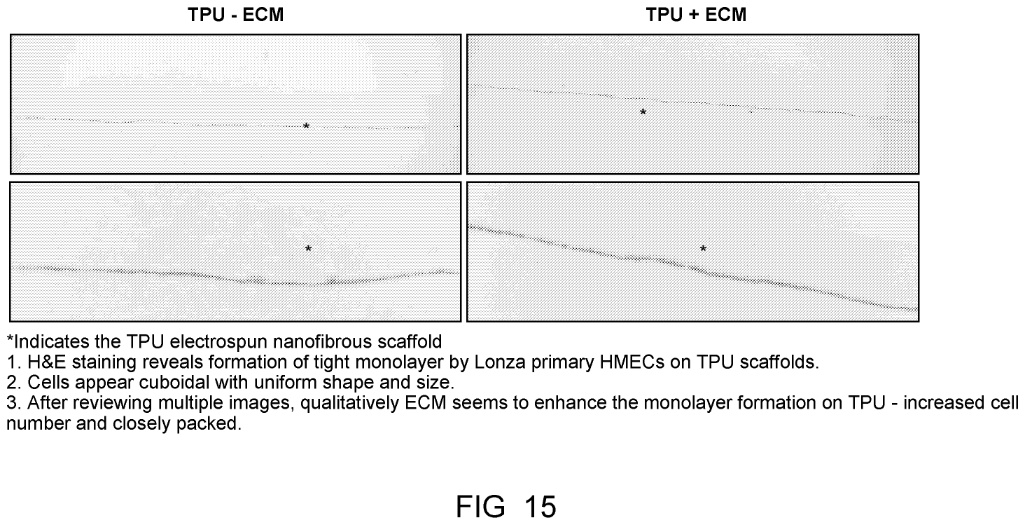

To further probe into the epithelial differentiation, the pHMECs were also stained for E-cadherin (an epithelial and tight junction marker). Positive E-cadherin staining was observed in Lonza pHMECs cultured on TPU the electrospun fibrous scaffolds, both with or without Collagen-IV and Laminin-1, (FIG. 9B). However, a membrane localization of E-cadherin (which is typically indicative of tight junction formation) was not observe in Lonza pHMECs on TPU the electrospun fibrous scaffolds (FIG. 9B). Nonetheless, a positive E-cadherin staining in Lonza pHMECs suggests the potential of the electrospun fibrous scaffolds to drive epithelial differentiation in Lonza pHMECs. In Sigma pHMECs cultured on the TPU electrospun fibrous scaffolds, both with or without Collagen-IV and Laminin-1, positive E-cadherin staining was observed along with its localization at cell membrane (indicated by white arrows) indicating epithelial differentiation as well as tight junction formation (FIG. 12). Sectioning of the cell laden TPU fibrous scaffold followed by H&E staining revealed the intact monolayer formed by Lonza pHMECs, which further confirmed the monolayer formation (FIG. 15). H&E staining also revealed the cuboidal shape and uniform size of pHMECs on the TPU fibrous scaffold which is again an indicative of epithelial traits (FIG. 15).

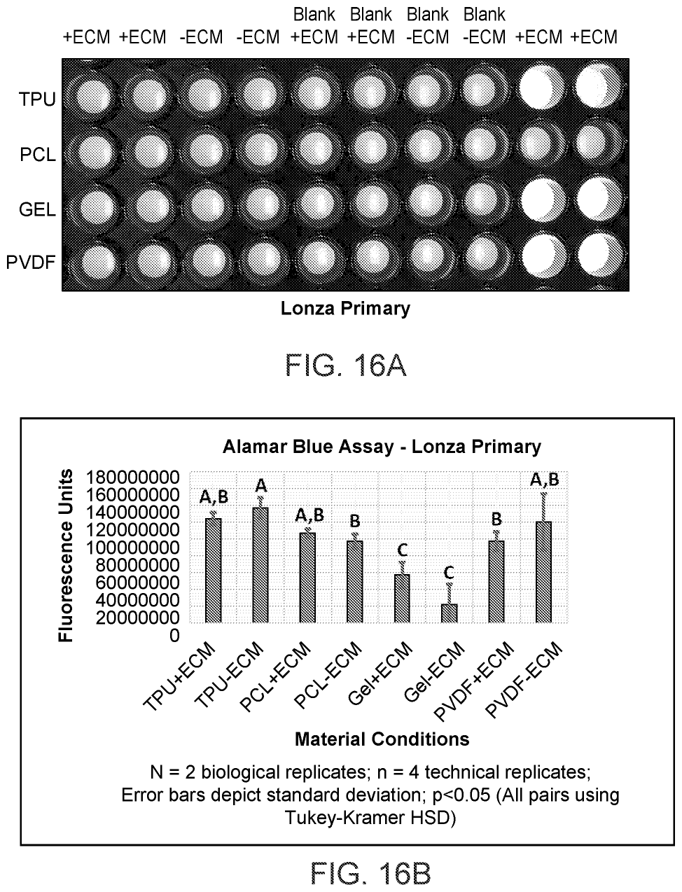

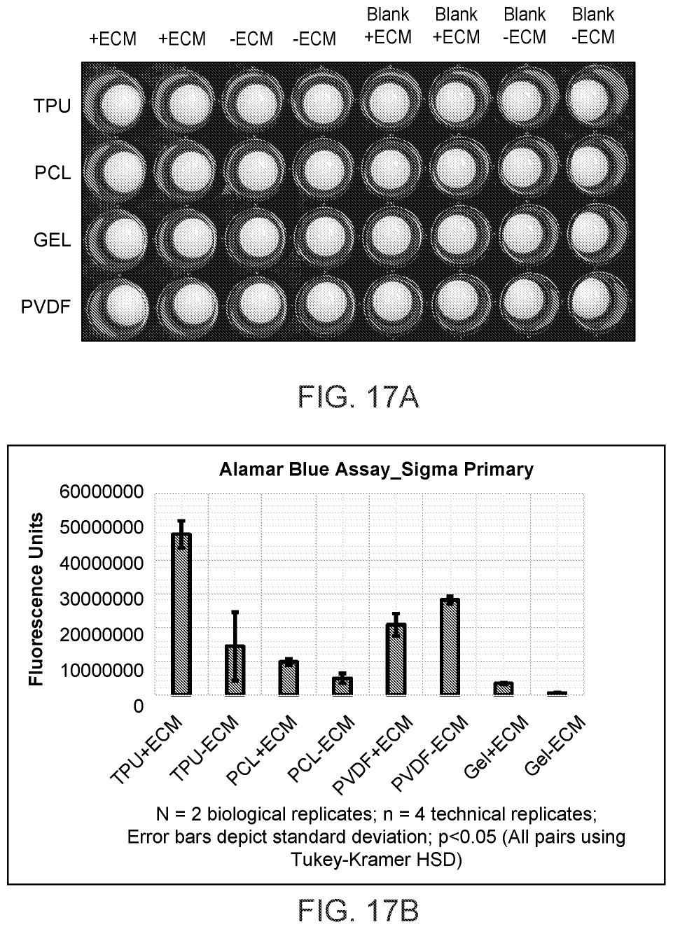

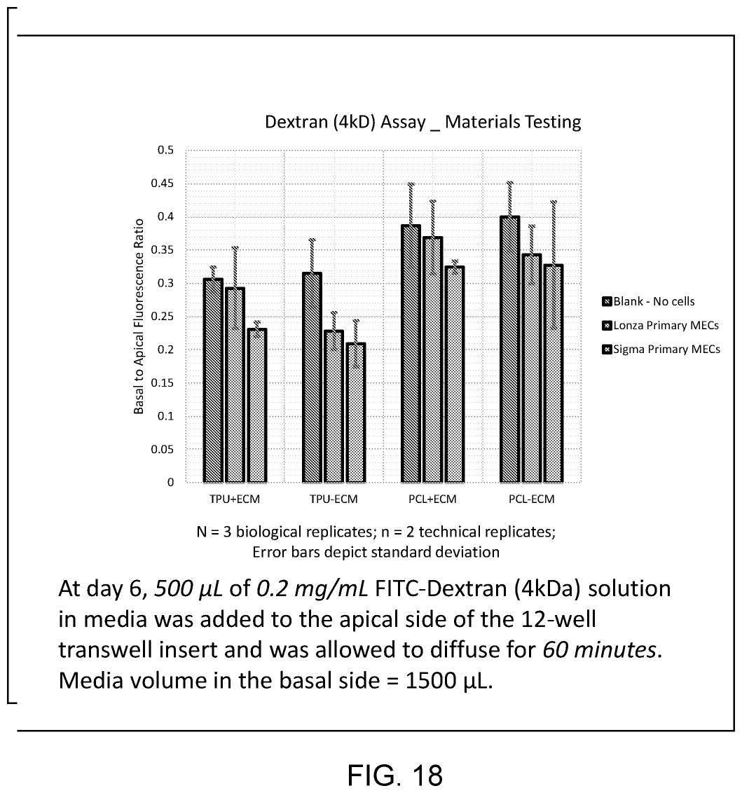

Further, an investigation into the proliferation of pHMECs on the TPU materials through Alamar blue assay at day 6 indicated a consistently higher proliferation on pHMECs on the TPU fibrous scaffolds (comprising the plurality of nanofibers) compared to PCL fibrous scaffolds (comprising a plurality of nanofibers) (FIGS. 16A-17B). Further, the monolayer barrier function was investigated through performing FITC-dextran (4 kD) diffusion assay. A decrease in the basal to apical fluorescence ratio was observed in cell laden TPU and PCL scaffolds compared to the blank TPU and PCL scaffold control which indicates that the cellular monolayer does exhibit some barrier functions in vitro like their actual physiological behavior (FIG. 18).

Accordingly, these preliminary results indicate the potential of basement membrane mimetic electrospun fibrous scaffolds (comprising a plurality of nanofibers) to support and sustain crucial biological processes underlying normal mammary epithelial cell behavior such as formation of epithelial monolayer, epithelial differentiation, tight junction formation, and epithelial barrier function. These epithelial biological processes regulated by basement membrane mimetic scaffolds can be synergistically attributed to their characteristics such as topographical features, mechanical stiffness, surface properties/chemistries which may recapitulate the bio-physical features of the physiological BM.

### Example 3: Mammary Epithelial Cell Growth Using Fibrous Scaffolds Comprising Polycaprolactone (PCL)

Cell growth, specifically primary human mammary epithelial cells (pHMECs), was demonstrated with synthetic fibrous scaffolds comprising a plurality of nanofibers (as described herein) that comprise polycaprolactone (PCL). The electrospun materials were made into sheets that were manually immobilized into a transwell insert (Scaffdex CellCrownTM inserts) for cell culture. Specifically, pHMECs sourced from Lonza and Sigma respectively were cultured for 6 days onto the electrospun fibrous scaffolds (comprising the plurality of nanofibers). Some of the scaffolds were coated with Collagen-IV and Laminin-1, and some of the scaffolds were not coated with Collagen-IV and Laminin-1. The cultured cells were probed for the following biological processes: a) cellular architecture/epithelial monolayer formation through actin immunofluorescence (IF) staining and hematoxylin and eosin (H&E) staining, b) tight junction formation/epithelial differentiation through IF staining for epithelial/tight junction marker E-cadherin staining, c) cell proliferation, and d) epithelial barrier function through FITC-dextran (4 kD) diffusion assay.

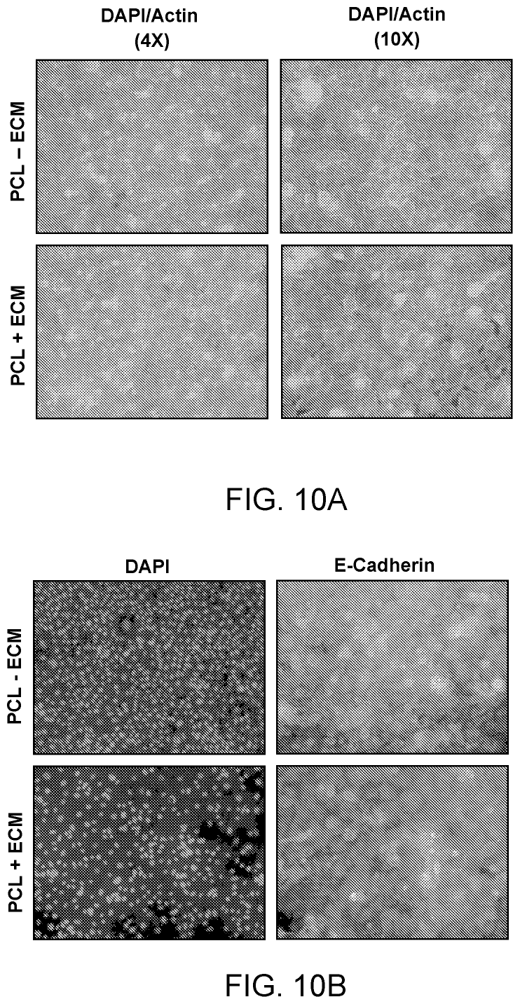

It was observed that Lonza pHMECs form intact cell monolayers on the electrospun fibrous scaffolds (comprising the plurality of nanofibers) made from PCL polymers, both with or without Collagen-IV and Laminin-1, as indicated by actin IF staining (FIG. 10A). The actin staining in Lonza pHMECs also revealed the presence of cortical actin which is indicative of an epithelial phenotype (FIG. 10A). As described in Example 4, contrary to Lonza pHMECs cultured on the electrospun fibrous scaffolds, the Lonza pHMECs did not form intact monolayers when cultured on conventional plastic as revealed by actin staining (at day 3) (FIG. 11A). The cells appeared to be non-uniform in shape and had a more spread morphology with formation of actin stress fibers which indicate deviation away from the epithelial phenotype.

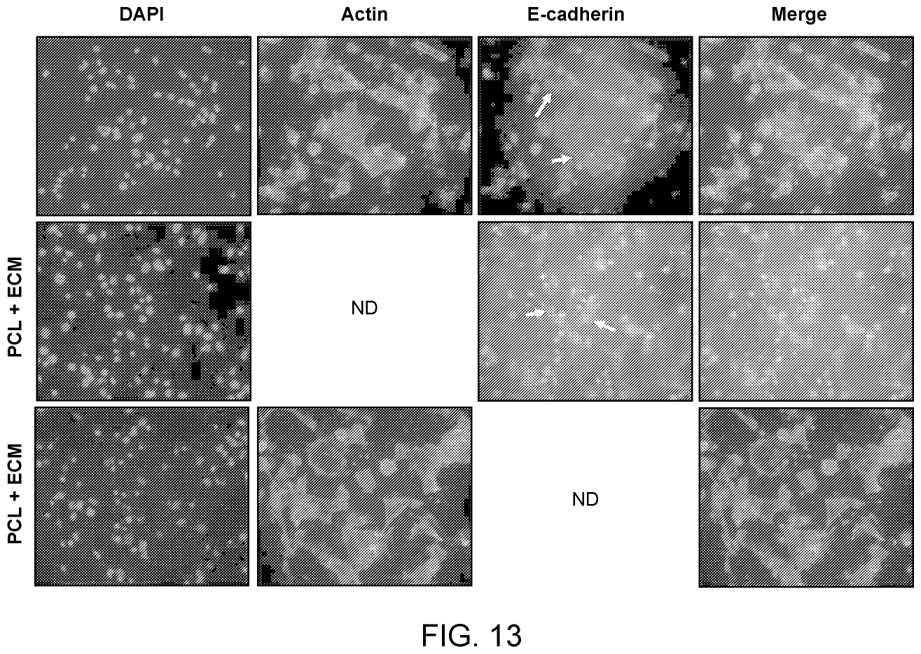

It was further observed that Sigma pHMECs do not form monolayers on the PCL electrospun fibrous scaffolds, however, they do grow in a form of isolated cluster/islands which suggests an epithelial phenotype (FIG. 13). This may also be attributed to the slower proliferation rate of Sigma pHMECs on PCL electrospun fibrous scaffolds, and hence, they may exhibit monolayer at a later time point compared to TPU electrospun fibrous scaffolds (as described herein, comprising a plurality of nanofibers). As described in Example 2, unlike Lonza pHMECs, Sigma pHMECs also form monolayer-like structures exhibiting cortical actin when cultured on conventional plastic (FIG. 14).

To further probe into the epithelial differentiation, the pHMECs were also stained for E-cadherin (an epithelial and tight junction marker). Positive E-cadherin staining was observed in Lonza pHMECs cultured on the PCL electrospun fibrous scaffolds, both with or without Collagen-IV and Laminin-1, (FIG. 10B). However, membrane localization of E-cadherin (which is typically indicative of tight junction formation) was not observed in Lonza pHMECs on the PCL electrospun fibrous scaffolds (FIG. 10B). Nonetheless, a positive E-cadherin staining in Lonza pHMECs does suggest the potential of electrospun fibrous scaffolds to drive epithelial differentiation in Lonza pHMECs.

Although Sigma pHMECs did not exhibit monolayer formation at day 6 timepoint on the PCL electrospun fibrous scaffolds, they did stain positive for E-cadherin and even exhibited a cell membrane localization of E-cadherin (indicated by white arrows), indicative of epithelial differentiation and tight junction formation (FIG. 13).

Further, an investigation into the proliferation of pHMECs on the PCL fibrous materials through Alamar blue assay at day 6 indicated a consistently higher proliferation on pHMECs on TPU fibrous scaffolds compared to PCL fibrous scaffolds (FIGS. 16A-17B). This might explain why Sigma pHMECs did not exhibit a monolayer on the PCL fibrous scaffolds at that timepoint. Further, the monolayer barrier function was investigated through performing FITC-dextran (4 kD) diffusion assay. A decrease in the basal to apical fluorescence ratio was observed in cell laden TPU and PCL fibrous scaffolds compared to the blank TPU and PCL fibrous scaffold control, which indicates that the cellular monolayer does exhibit some barrier functions in vitro like their actual physiological behavior (FIG. 18).

Accordingly, these preliminary results indicate the potential of basement membrane mimetic electrospun fibrous scaffolds (comprising a plurality of nanofibers) to support and sustain crucial biological processes underlying normal mammary epithelial cell behavior such as formation of epithelial monolayer, epithelial differentiation, tight junction formation, and epithelial barrier function. These epithelial biological processes regulated by basement membrane mimetic fibrous scaffolds can be synergistically attributed to their characteristics such as topographical features, mechanical stiffness, surface properties/chemistries which may recapitulate the bio-physical features of the physiological BM.

### Example 4: Characteristics of PCL and TPU scaffolds

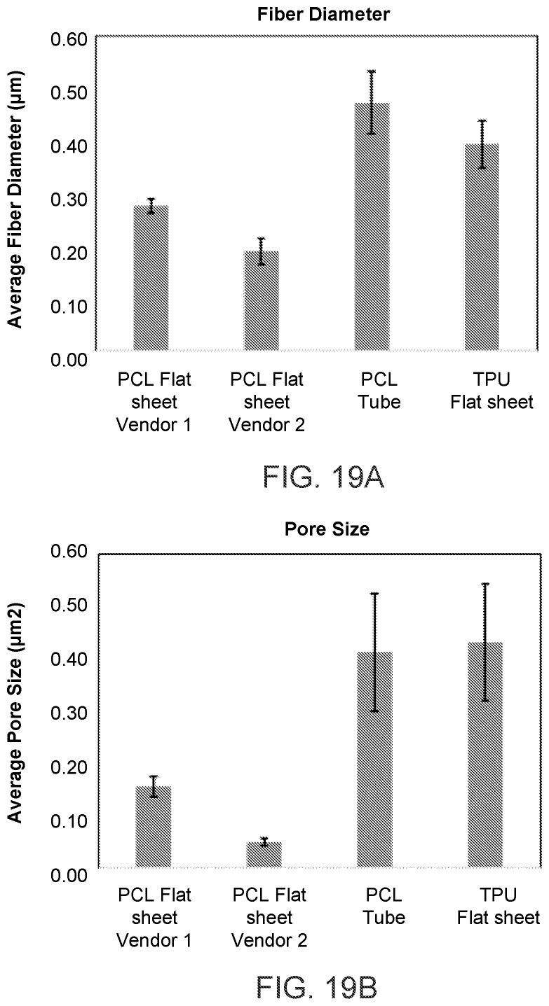

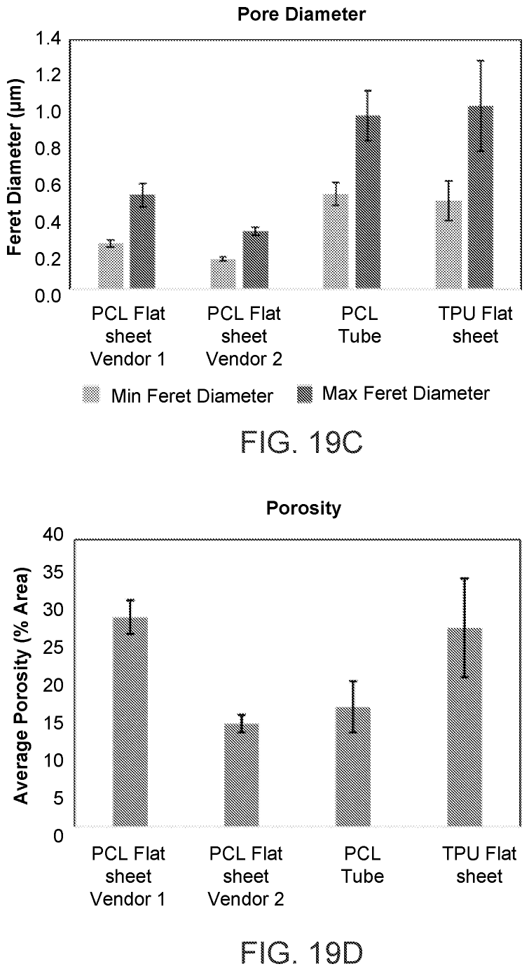

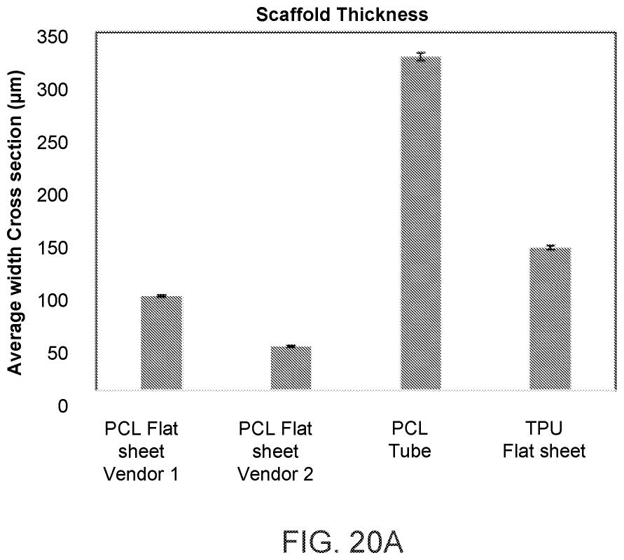

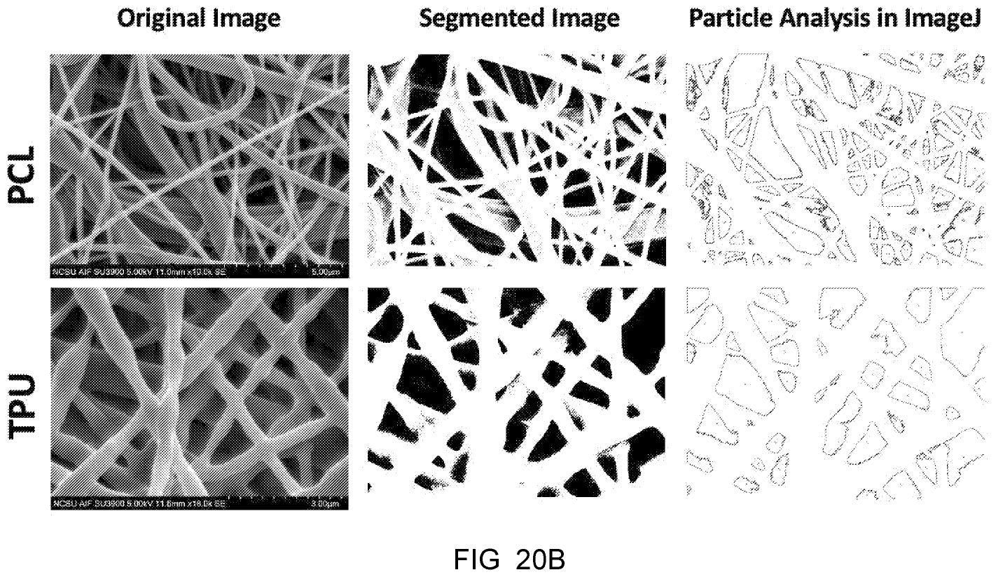

Scaffolds fabricated from PCL were prepared as flat sheet or as tube structures, whereas TPU scaffolds were only evaluated as flat sheets. Tube structures can be incorporated into hollow fiber bioreactor cartridges to achieve production-scale culture, whereas flat sheets can be incorporated into other bioreactor designs. Both materials are composed of nanofiber structures. Based on analysis of scanning electron micrographs (SEM) of each material, a variety of physical properties was measured, including the average diameter of the nanofibers that compose the material, pore size (measured as area, μ^{m}2), pore diameter, and average porosity (FIGS. 19A-19D). Flat sheets of PCL obtained from 2 different vendors were similar with respect to nanofiber diameter, pore size, and pore diameter; however they differed with regard to porosity. Porosity was highest for PCL flat sheets from vendor 1, and for TPU. The thickness of each scaffold was measured and is showed that PCL fabricated into tubes had the highest thickness (FIG. 20A). Examples of SEM images of the PCL and TPU materials, image segmentation, and the particle analysis that was applied to identify pores for measurement are also provided (FIG. 20B). Young's Modulus and hydrophobicity was measured and the results show that these values were similar across all materials.

### Response of Modified Human MECs to PCL and TPU Scaffolds

PCL and TPU were identified as scaffold materials to support adherent MEC culture for the bioproduction of milk products based on the morphological and growth characteristics of commercially available primary human MECs (FIGS. 9-18). This example describes the cellular response to these materials to include human MECs that have been modified for extended lifespan in culture.

The behaviors and phenotypes of primary cells isolated from living tissue are affected by growth in culture, and primary cells will generally undergo rapid senescence when removed from the tissue microenvironment. Recapitulation of a complex process such as milk biosynthesis at physiological scale requires that cells replicate through many generations in culture while preserving their capacity to achieve functional phenotypes. Specific genetic modifications of primary MECs to circumvent the stress-induced and replicative senescence that are typical of primary cell culture can enable production-scale culture, however optimization of the biophysical environment is important to guide and maintain the appropriate cellular phenotypes to achieve production. In particular, for milk biosynthesis, the mammary epithelium must be able to form and maintain a barrier between the source of nutrients and the compartment into which milk is collected.

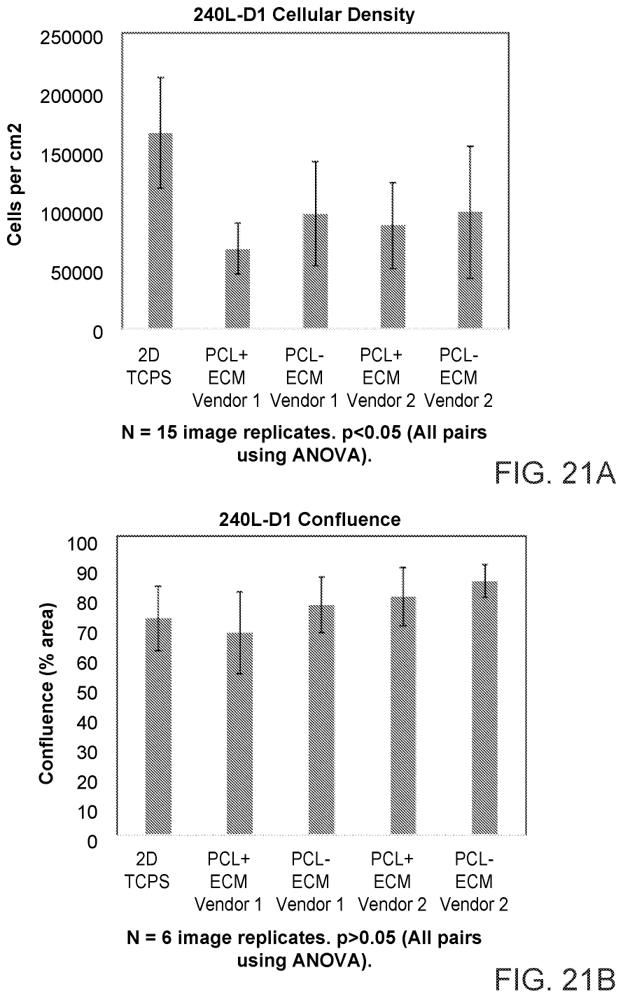

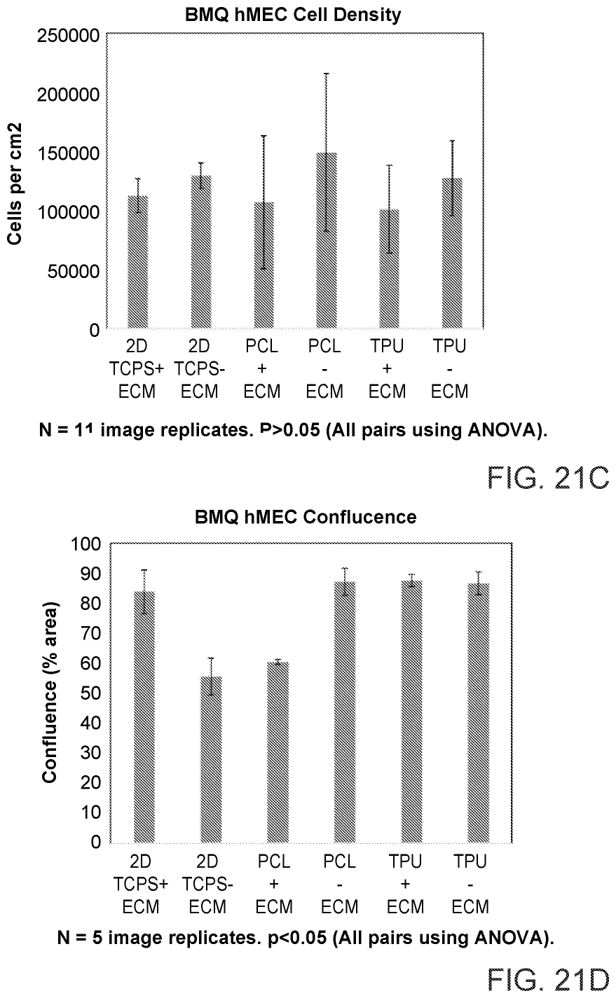

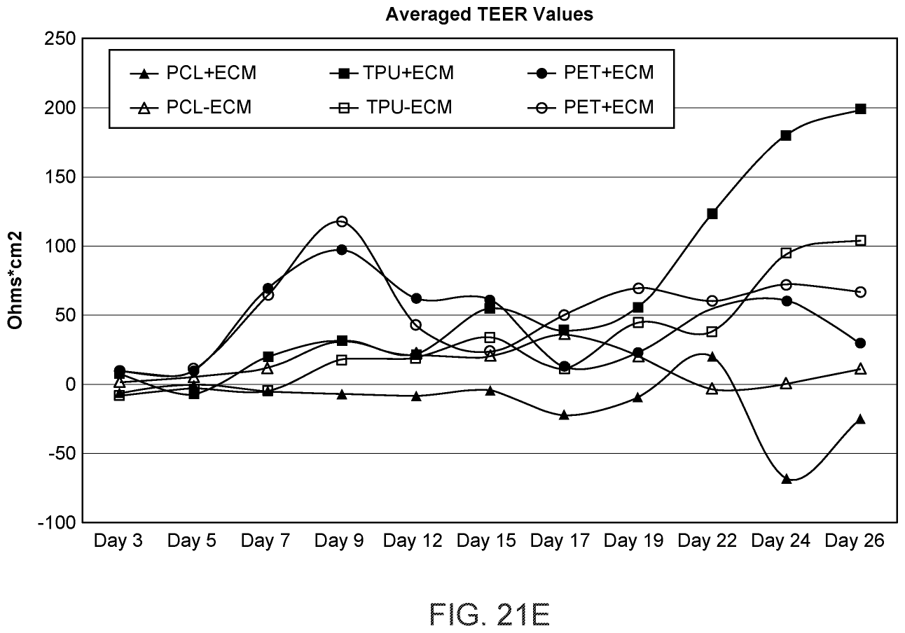

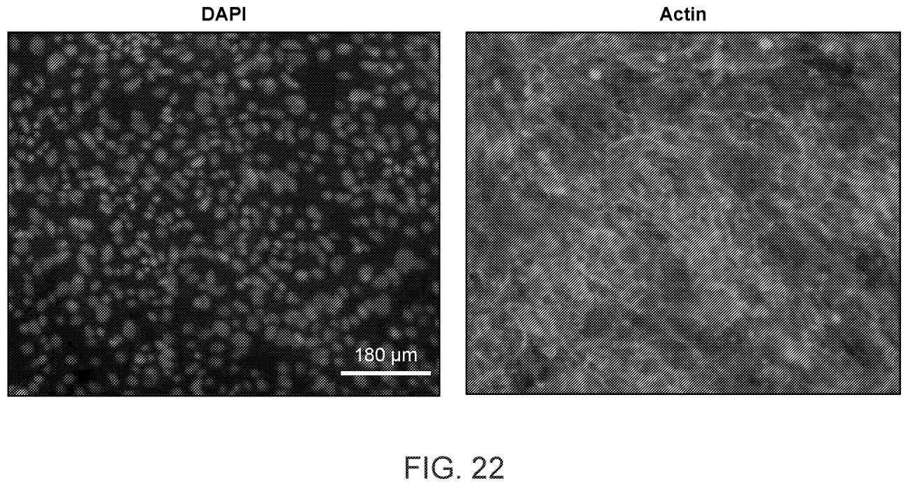

PCL and TPU were assessed for potential scaffolds to support the production of cell cultured milk, and the response of several human MEC lines that have been modified for extended lifespan to these materials was evaluated. Specifically, cell density and degree of confluence were examined using a publicly available hMEC line (240L-D1) and a proprietary BIOMILQ hMEC line (BMQ) FIGS. 21A-21D).

Each hMEC line was cultured on either PCL or TPU that had been either coated or uncoated with an extracellular matrix (ECM) to facilitate cell adhesion. Cell density was measured using an automated image analysis to calculate the number of DAPI-stained nuclei per cm² (FIG. 22, left panel). Confluence was measured by analyzing images of cells stained for actin (FIG. 22, right panel), which delineates the cell periphery. The results show, that PCL and TPU both supported similar cell density and confluence compared to tissue culture polystyrene, used as a control.

The barrier function of epithelial monolayers prepared by growing 240L-D1 or BMQ hMECs in Transwells was examined (FIG. 21E). Transepithelial electrical resistance (TEER) analysis revealed that hMECs grown on ECM-coated TPU Transwells achieved higher TEER readings than any of the other conditions tested, indicating improved barrier function.

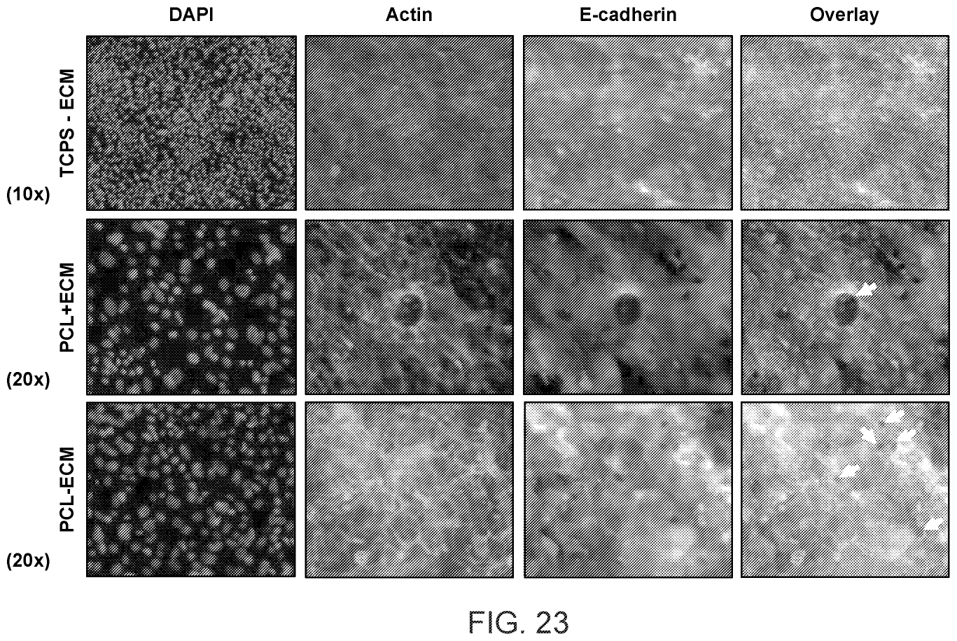

Finally, ECM-coated PCL, and to a lesser extent TPU, supported the self-organization of 240L-D1 and BMQ hMECs into distinct structures that appeared as void areas reminiscent of the lumen of the mammary acini (FIG. 23). When examined by fluorescence microscopy for nuclei, actin, and E-cadherin, distinct localization patterns were observed around these structures. In particular, nuclei were absent from the interior region, suggesting that these structures are hollow. Additionally, the structures were surrounded by strong staining for filamentous actin and increased levels of E-cadherin. These features are consistent with apical orientation of actin and E-cadherin and suggest polarization of cellular machinery as required for milk secretion. Notably, these structures were not observed when the same cell lines were grown on tissue culture polystyrene, and they were more prevalent when grown on ECM-coated materials than on uncoated materials.

Taken together, the results described show that PCL and TPU represent preferred materials for use as scaffolds for the adherent culture of MECs for the synthesis of cell cultured milk products.

### Methods

### Analysis of the Physical Characteristics of TPU and PCL

Fiber diameter and orientation. Scanning electron microscopy images were taken of a series of TPU and PCL samples to visualize their micro-scale surface structure and analyzed to quantify key properties of each scaffold. To assess fiber diameter, these images were then converted to binary and segmented via SIMPoly MATLAB code. This method was validated via a series of manual measurements using open-sans ImageJ software. ImageJ plug-in DiameterJ was used to quantify degree of alignment of scaffold fibers within these images proving random alignment of said fibers as expected during manufacture.

Scaffold thickness. Portions of these scaffolds were sliced to produce cross-sections and imaged via scanning electron microscopy. Using ImageJ, a series of manual measurements taken tangentially to the flat scaffold cross-sections quantified average scaffold thickness for each material.

Young's Modulus. Samples of both PCL and TPU were tested using an Instron universal tensile machine at matching speeds to define elasticity via Young's Modulus.

Porosity and pore size. Scanning electron microscopy images used to assess fiber diameter and orientation were also used to quantify porosity and pore size. Similarly, each image was segmented via ImageJ and analyzed with the Analyze Particles tool. Percent area and particle size were used to define porosity and pore size, respectively.

Hydrophobicity. To quantify hydrophobicity of PCL and TPU, a series of scaffolds were placed flat on a stage and an ultrapure droplet of water was dispensed at the scaffold edge illuminated with a diffused backlight. A macro lens placed horizontal to the plane then immediately took an image of the droplet cross-section. Placement of droplets and imaging were repeated at various edges of each sample. Each image was then analyzed via ImageJ's DropSnake plug-in to produce contact angle values.

Modified human MECs cultured on TPU and PCL

Morphological observations. A series of experiments involved seeding various hMECs on TPU and PCL and allowed the cells to proliferate. At termination of each experiment, cell-laden scaffolds were fixed and stained for DAPI, actin, and E-cadherin to assess cell density, confluence and tight junction formation, respectively. Fluorescent images were taken of each scaffold under the appropriate channels and analyzed with ImageJ. Each image was segmented and quantified using the Particle Analysis tool producing nuclei count via DAPI and percent coverage of the total area via actin stain. Overlay images were produced with DAPI, actin, and E-cadherin images to visualize cell architecture. From these images, there was an observed prevalence of acinar (hollow lumen) structures on TPU and PCL scaffolds suggesting lactogenic phenotypes.

Barrier function (TEER). Barrier function and epithelial monolayer formation were assessed over time via transepithelial electrical resistance (TEER) chopstick assay. Custom PCL and TPU transwell inserts were first constructed. Cells were then grown on these scaffold inserts with and without extracellular matrix coating in conjunction with matching PET transwell controls and appropriate acellular blank inserts. TEER measurements were taken every two days and subtracted by the blank values to quantify compartmentalization.

The foregoing examples are illustrative of the present disclosure and are not to be construed as limiting thereof. Although the disclosure has been described in detail with reference to preferred embodiments, variations and modifications exist within the scope and spirit of the disclosure as described and defined in the following claims.

What is claimed is:

1. A cell construct, comprising:
a. a three dimensional scaffold comprising a plurality of fibers that are non-uniformly oriented and/or non-linearly oriented and that comprise thermoplastic polyurethane and/or polycaprolactone, said three dimensional scaffold having an exterior surface, an interior surface defining an interior cavity/basal chamber, said three dimensional scaffold being at least partially permeable from the interior surface to the exterior surface;
b. a culture media disposed within the interior cavity/basal chamber and in fluidic contact with the internal surface; and
c. an at least partially confluent monolayer of polarized mammary cells coupled to the exterior surface of the three-dimensional scaffold, or a portion thereof, wherein the mammary cells comprise mammary epithelial cells, mammary myoepithelial cells, and/or mammary progenitor cells.

2. The cell construct of claim 1, wherein the polarized mammary cells comprise an apical surface and a basal surface.

3. The cell construct of claim 1, wherein the basal surface of the mammary cells is in fluidic contact with the culture media.

4. The cell construct of claim 1, wherein the three dimensional scaffold is configured to mimic a basement membrane of a mammary gland based on a specified set of one or more features for said three dimensional scaffold.

5. The cell construct of claim 2, wherein the one or more features comprise one or more topological features, one or more mechanical properties, one or more surface properties, one or more viscoelastic properties, or a combination thereof.

6. The cell construct of claim 5, wherein the one or more topological features comprise i) an average fiber diameter of the plurality of fibers, ii) orientation(s) of the plurality of fibers, or iii) a combination thereof.

7. The cell construct of claim 6, wherein the average fiber diameter is from about 5 nm to about 5000 nm, from about 5 nm to about 50 nm, from about 50 nm to about 150 nm, from about 100 nm to about 300 nm, from about 100 nm to about 500 nm, from about 200 nm to about 1000 nm, from about 500 nm to about 1500 nm, from about 1000 nm to about 3000 nm, or from about 1500 nm to about 5000 nm.

8. The cell construct of claim 5, wherein the one or more mechanical properties comprises i) a thickness of the three dimensional scaffold, ii) a modulus of elasticity of the three dimensional scaffold, iii) a permeability of the three dimensional scaffold, or iv) a combination thereof.

9. The cell construct of claim 8, wherein the thickness of the three dimensional scaffold is from about 20 μm to about 100 μm.

10. The cell construct of claim 8, wherein the modulus of elasticity of the three dimensional scaffold is from about 100 Pa to about 300 Pa.

11. The cell construct of claim 8, wherein the three dimensional scaffold comprises a plurality of pores extending from the interior surface to the exterior surface, thereby enabling said permeability.

12. The cell construct of claim 11, wherein the plurality of pores define corresponding channel(s) that pass through the three dimensional scaffold.

13. The cell construct of claim 11, wherein the permeability of the three dimensional scaffold correlates to a porosity of the three dimensional scaffold, wherein the porosity is from about 5% to about 95%, from about 15% to about 75%, from about 25% to about 70%, or from about 40% to about 60%.

14. The cell construct of claim 11, wherein the plurality of pores have an average maximum dimension across the exterior surface from about 5 nm to about 1000 nm, from about 5 nm to about 50 nm, from about 50 nm to about 150 nm, from about 100 nm to about 500 nm, or from about 250 nm to about 1000 nm.

15. The cell construct of claim 11, wherein the plurality of pores have an average maximum dimension across the exterior surface from about 8 nm to about 10 nm, from about 25 nm to about 75 nm, from about 100 nm to about 250 nm, from about 200 nm to about 400 nm, or from about 300 nm to about 600 nm.

16. The cell construct of claim 5, wherein one or more of the fibers comprises one or more polymer chains of a polymer material.

17. The cell construct of claim 16, wherein the polymer material comprises thermoplastic polyurethane and/or polycaprolactone.

18. The cell construct of claim 16, wherein the one or more viscoelastic properties of the scaffold is based on a degree of entanglement of a polymer chain of the fibers.

19. The cell construct of claim 18, wherein the one or more viscoelastic properties of the scaffold is based on a ratio of a degree of entanglement of a polymer chain of the fibers with itself to a degree of entanglement of two or more polymer chains of the fibers.

20. The cell construct of claim 18 or 19, wherein the degree of entanglement is determined via the Gauss Linking Integral.

21. The cell construct of claim 5, wherein the one or more surface properties comprises i) a specific surface area of the three dimensional scaffold, ii) specified hydrophobicity and/or hydrophilicity at specified region(s) of the three dimensional scaffold, iii) a surface charge of the three dimensional scaffold, iv) one or more surface coatings applied to the three dimensional scaffold, v) an extent of the one or more surface coatings, or vi) a combination thereof.

22. The cell construct of claim 21, wherein the hydrophobicity and/or hydrophilicity of the three dimensional scaffold is based on a surface treatment applied to the three dimensional scaffold.

23. The cell construct of claim 22, wherein the surface treatment includes plasma treatment.

24. The cell construct of claim 21, wherein the surface charge of the three dimensional scaffold is based on a surface treatment applied to the three dimensional scaffold.

25. The cell construct of claim 24, wherein the surface treatment includes poly-l-lysine coating to make surface more positively charged for cell attachment, and/or coating with mussel-inspired adhesive L-DOPA for enhanced cell attachment.

26. The cell construct of claim 21, wherein the one or more surface coatings comprise a matrix material.

27. The cell construct of claim 26, wherein the matrix material comprises one or more extracellular matrix proteins.

28. The cell construct of claim 26, wherein the matrix material comprises Collagen-IV, Laminin-1, RGD peptide, laminin peptides like IKVAV, other ECM-peptides, or a combination thereof.

29. The cell construct of claim 1, wherein the exterior surface is uncoated.

30. The cell construct of claim 1, wherein a population of the plurality of fibers are nanofibers.

31. The cell construct of claim 1, wherein the plurality of fibers are hollow.

32. The cell construct of claim 1, wherein the plurality of fibers are electrospun, wet spun, dry spun, melt spun, phase inversion spun, or a combination thereof.

33. The cell construct of claim 1, wherein the three dimensional scaffold is configured to activate a Jak2-Stat5 milk biosynthetic pathway via the mammary cells.

34. The cell construct of claim 1, wherein at least 70%, at least 80%, at least 90%, at least 95%, at least 99%, or 100% of the mammary cells are polarized in the same orientation.

35. The cell construct of claim 1, wherein the monolayer of polarized mammary cells is at least 70% confluent, at least 80% confluent, at least 90% confluent, at least 95% confluent, at least 99% confluent, or 100% confluent.

36. The cell construct of claim 1, wherein the mammary cells comprise a constitutively active prolactin receptor protein.

37. The cell construct of claim 1, wherein the culture medium comprises prolactin.

38. The cell construct of claim 1, wherein the three dimensional scaffold comprises a sheet configuration, a mat configuration, a sphere configuration, or a tube configuration.

39. The cell construct of claim 38, wherein the tube configuration defines one or more conduits.

40. The cell construct of claim 38, wherein the mat configuration is configured to be folded so as to form the tube configuration.

41. A method of producing an isolated cultured milk product from mammary cells, the method comprising:
a. culturing a cell construct in a bioreactor under conditions which produce the cultured milk product, said cell construct comprising:
i. a three dimensional scaffold comprising a plurality of fibers that are non-uniformly oriented and/or non-linearly oriented and that comprise thermoplastic polyurethane and/or polycaprolactone, said three dimensional scaffold having an exterior surface, an interior surface defining an interior cavity/basal chamber, said three dimensional scaffold being at least partially permeable from the interior surface to the exterior surface;
ii. a culture media disposed within the interior cavity/basal chamber and in fluidic contact with the internal surface; and
iii. an at least partially confluent monolayer of polarized mammary cells coupled to the exterior surface of the three-dimensional scaffold, or a portion thereof, wherein the mammary cells comprise mammary epithelial cells, mammary myoepithelial cells, and/or mammary progenitor cells; and
b. isolating the cultured milk product.

42. The method of claim 41, comprising the cell construct of any one of claims 2 to 40.

43. The method of claim 41, wherein the bioreactor comprises an apical compartment that is substantially isolated from the internal cavity of the cell construct.

44. The method of claim 41, wherein a basal surface of the mammary cells is in fluidic contact with the culture media.

45. The method of claim 43, wherein the apical compartment is in fluidic contact with an apical surface of the mammary cells.

46. The method of claim 45, wherein the cultured milk product is secreted from the apical surface of the mammary cells into the apical compartment.

47. The method of claim 41, wherein the cell construct further comprises a plurality of plasma cells disposed on the exterior surface.

48. The method of claim 47, wherein the cultured milk product comprises secretory IgA (sIgA) and/or IgG.

49. The method of claim 47, wherein total cell density of plasma cells in the bioreactor is about 200 to 500 plasma cells per mm².

50. The method of claim 41, wherein the culture media substantially does not contact the cultured milk product.

51. The method of claim 41, wherein total cell density of mammary cells within the bioreactor is at least 10¹¹; and alternatively wherein total surface area of mammary cells within the bioreactor is at least about 450 cm² or at least about 1.5 m².

52. The method of claim 41, wherein the culturing is carried out at a temperature of about 27° C. to about 39° C.

53. The method of claim 41, wherein the culturing is carried out at an atmospheric concentration of CO² of about 4% to about 6%.

54. A bioreactor, comprising:
a. an apical compartment comprising a cultured milk product; and
b. at least one live cell construct comprising:
i. a three dimensional scaffold comprising a plurality of fibers that are non-uniformly oriented and/or non-linearly oriented and that comprise thermoplastic polyurethane and/or polycaprolactone, said three dimensional scaffold having an exterior surface, an interior surface defining an interior cavity/basal chamber, said three dimensional scaffold being at least partially permeable from the interior surface to the exterior surface;
ii. a culture media disposed within the interior cavity/basal chamber and in fluidic contact with the internal surface; and
iii. an at least partially confluent monolayer of polarized mammary cells coupled to the exterior surface of the three-dimensional scaffold, or a portion thereof, wherein the mammary cells comprise mammary epithelial cells, mammary myoepithelial cells, and/or mammary progenitor cells.

55. The bioreactor of claim 54, comprising the cell construct of any one of claims 2 to 40.

56. The bioreactor of claim 54, wherein the total cell density of mammary cells within the bioreactor is at least 10¹¹.

57. The bioreactor of claim 54, wherein the total surface area of mammary cells within the bioreactor is at least about 450 cm² or at least about 1.5m².

58. A method for producing a scaffold for isolated cultured milk production from mammary cells, the method comprising
a. forming a porous mat comprising a plurality of fibers that are non-uniformly oriented and/or non-linearly oriented, said fibers comprising thermoplastic polyurethane and/or polycaprolactone.

59. The method of claim 58, further comprising folding the porous mat into a tubular configuration.

60. The method of claim 58, wherein forming the porous mat comprises electrospinning, wet spinning, dry spinning, melt spinning, and/or phase inversion spinning.

61. The method of claim 58, wherein the mat comprises an exterior surface, an interior surface defining an interior cavity/basal chamber, and a plurality of pores extending from the interior surface to the exterior surface.

62. The method of claim 58, wherein forming the porous mat creates a specified set of one or more features for said scaffold.

63. The method of claim 62, wherein the one or more features comprise one or more topological features, one or more mechanical properties, one or more surface properties, one or more viscoelastic properties, or a combination thereof.

64. The method of claim 63 wherein the one or more topological features comprise i) an average fiber diameter of the plurality of fibers, ii) orientation(s) of the plurality of fibers, or iii) a combination thereof.

65. The method of claim 64, wherein the average fiber diameter is from about 5 nm to about 5000 nm, from about 5 nm to about 50 nm, from about 50 nm to about 150 nm, from about 100 nm to about 300 nm, from about 100 nm to about 500 nm, from about 200 nm to about 1000 nm, from about 500 nm to about 1500 nm, from about 1000 nm to about 3000 nm, or from about 1500 nm to about 5000 nm.

66. The method of claim 63, wherein the one or more mechanical properties comprises i) a thickness of the three dimensional scaffold, ii) a modulus of elasticity of the three dimensional scaffold, iii) a porosity of the three dimensional scaffold, or iv) a combination thereof.

67. The method of claim 66, wherein the thickness of the scaffold is from about 20 μm to about 100 μm.

68. The method of claim 66, wherein the modulus of elasticity of the scaffold is from about 100 Pa to about 300 Pa.

69. The method of claim 66, wherein the porosity of the scaffold is from about 5% to about 95%, from about 15% to about 75%, from about 25% to about 70%, or from about 40% to about 60%.

70. The method of claim 66, wherein the plurality of pores have an average maximum dimension across the exterior surface from about 5 nm to about 1000 nm, from about 5 nm to about 50 nm, from about 50 nm to about 150 nm, from about 100 nm to about 500 nm, or from about 250 nm to about 1000 nm.

71. The method of claim 66, wherein the plurality of pores have an average maximum dimension across the exterior surface from about 8 nm to about 10 nm, from about 25 nm to about 75 nm, from about 100 nm to about 250 nm, from about 200 nm to about 400 nm, or from about 300 nm to about 600 nm.

72. The cell construct of claim 63, wherein one or more of the fibers comprises one or more polymer chains of a polymer material.

73. The cell construct of claim 72, wherein the polymer material comprises thermoplastic polyurethane and/or polycaprolactone.

74. The cell construct of claim 72, wherein the one or more viscoelastic properties of the scaffold is based on a degree of entanglement of a polymer chain of the fibers.

75. The cell construct of claim 74, wherein the one or more viscoelastic properties of the scaffold is based on a ratio of a degree of entanglement of a polymer chain of the fibers with itself to a degree of entanglement of two or more polymer chains of the fibers.

76. The cell construct of claim 74 or 75, wherein the degree of entanglement is determined via the Gauss Linking Integral.

77. The method of claim 63, wherein the one or more surface properties comprises i) a specific surface area of the scaffold, ii) specified hydrophobicity and/or hydrophilicity at specified region(s) of the scaffold, iii) a surface charge of the scaffold, iv) one or more surface coatings applied to the scaffold, v) an extent of the one or more surface coatings, or vi) a combination thereof.

78. The method of claim 77, wherein the hydrophobicity and/or hydrophilicity of the scaffold is based on a surface treatment applied to the scaffold.

79. The method of claim 78, wherein the surface treatment includes plasma treatment.

80. The method of claim 77, wherein the surface charge of the scaffold is based on a surface treatment applied to the scaffold.

81. The method of claim 80, wherein the surface treatment includes poly-l-lysine coating to make surface more positively charged for cell attachment, and/or coating with mussel-inspired adhesive L-DOPA for enhanced cell attachment.

82. The method of claim 77, wherein the one or more surface coatings comprise a matrix material.

83. The method of claim 82, wherein the matrix material comprises one or more extracellular matrix proteins.

84. The method of claim 82, wherein the matrix material comprises Collagen-IV, Laminin-1, RGD peptide, laminin peptides like IKVAV, other ECM-peptides, or a combination thereof.

85. The method of claim 58, wherein the exterior surface is uncoated.

86. The method of claim 58, wherein a population of the plurality of fibers are nanofibers.

87. The method of claim 58, wherein the plurality of fibers are hollow.

88. A scaffold for isolated cultured milk production from mammary cells formed by any of the methods of claims 58-86.
